(19) United States
(12) Patent Application Publication
(10) Pub. No.: US 2025/0163508 A1
(43) Pub. Date: May 22, 2025

(54) ANALYSIS SYSTEM, PLATE, AND ANALYSIS METHOD

(71) Applicant: Konica Minolta, Inc., Tokyo (JP)

(72) Inventors: Mayuka KABASAWA, Tokyo (JP); Hiroshi KITA, Tokyo (JP); Shumpei HITOSUGI, Tokyo (JP); Takatoshi SUEMATSU, Tokyo (JP)

(21) Appl. No.: 18/843,058

(22) PCT Filed: Mar. 9, 2023
(86) PCT No.: PCT/JP2023/008996

§ 371 Date: Aug. 30, 2024

(30) Foreign Application Priority Data
Mar. 10, 2022 (JP) 2022-037178

(51) Int. Cl. C12Q 1/6876

(52) U.S. Cl. CPC C12Q 1/6876

(57) ABSTRACT

An object of the present invention is to provide an analysis system capable of analyzing a target substance more accurately, which includes: a plate including reaction fields for accommodating first and a second components, and partitioned at intervals; a signal information acquisition section to acquire first signal information from the plate and second signal information from the plate; and an analysis section for performing machine learning and analyzing a difference between the first signal information and the second signal information. At least two reaction fields respectively accommodate the first components having different compositions, and in at least one reaction field, reactions including interaction of the first component and/or the second component are caused.

### TECHNICAL FIELD

The present invention relates to an analysis system, a plate, and an analysis method.

### BACKGROUND ART

Conventionally, various types of information have often been output for human analysis and utilization. However, in recent years, digitalization has progressed and information has become more complicated. Then, for example, data that is more than three dimensional is output to a machine and is analyzed by the machine. That is, the output target changes from a human to a machine.

So far, in the flow of technology development and research and development, it has been the basis of research and development that researchers conceive laws and correlations from data obtained from experiments with a deductive logic as starting points, establish “hypotheses” conceived thereby, and verify (abduction) the hypotheses.

On the other hand, there is also a method of “inductive” interpretation as a counter point of “deductive” hypothesis planning. Data used in the inductive interpretation method is data that is difficult to handle a priori by human thinking, and is, for example, scientific data for which the basis is not sufficiently established, scientific data to which meaning is not sufficiently given, or the like. Furthermore, even scientific data for which the basis is sufficiently established or meaning is sufficiently provided, scientific data that is difficult to process a priori by human thought due to reasons such as complexity or a large amount of information is included in inductive data. Such inductive data is useful as data for machine learning, but generation of a large amount of data is a current rate-limiting factor.

Desirable requirements for such machine learning data include being multidimensional and a large amount of data, being able to be simply output, being linked to a substance or a state, and the like. For example, in chemical science, a wide variety of changes occur, such as formation of ionic bonds and coordinate bonds, state changes such as association, aggregation, and crystallization, and quantum state changes such as excitation, transition, and relaxation. Then, data obtained along with this is suitable as data for machine learning. In particular, information on light or color can be associated with a physical or quantum state change caused by reaction, interaction, complex formation, ionization, or the like of a substance. Further, there are various types of light and color measuring devices, and it is possible to acquire a large amount of multidimensional data and to perform simple output.

On the other hand, in bioscience, a high-throughput method using a sequencer, a plate reader, or the like has been widely used in recent years. In addition, automation using an automatic reactor or an automatic measuring machine is also progressing. In the above-described field, various feature amounts that are easy to measure can be obtained from a large amount of data generated, for example, by using pictorial imaging for machine learning. However, there are also problems such as low reproducibility.

In a high-throughput method, an analysis instrument is often used in which single-stranded DNA such as a DNA microarray is disposed on various substrates, reacted with a specimen having a complementary base sequence, and detected by fluorescence or electric current. This is a method of detecting a target substance by utilizing a specific interaction, and obtains information on a specific substance. On the other hand, when the reagents are mixed on the substrate, a reaction such as a more complicated chemical reaction, an ion reaction, a complex-forming reaction, an interaction, or energy transfer occurs, and data in which the feature amount related to the substance and the phenomenon is enhanced is obtained. However, the data generated by the reaction is often multidimensional data obtained by integrating a plurality of reactions, and may not be understood by humans. The multidimensional data as described above has been data that cannot be solved by conventional science that humans interpret, that is, data that does not make sense for humans, but in recent years, effectiveness has emerged through machine learning and deep learning by artificial intelligence (AI).

Therefore, in recent years, there has been a demand for providing a “reaction field” which has a plurality of reaction systems and in which a complicated interaction or chemical reaction occurs, and an analysis method using the same, which are applicable to such machine learning. According to such a “reaction field”, a large amount of multidimensional data can be generated.

For example, NPL 1 describes forming a two dimensional colorimetric sensor by overprinting a plurality of chemical reagents in a pattern at a high density using an inkjet printer. It is described that, after a sensor is produced, an analysis target is applied by a spray method, and the color of the sensor is subjected to machine learning. That is, in the above-described technique, each region of the colorimetric sensor functions as a type of reaction field.

Furthermore, PTL 1 describes analyzing, in a columnar graph, a result including a variety of information obtained using particles to which a plurality of different luminescent probes are bonded.

### CITATION LIST

### Non Patent Literature

NPL 1 Run Luo, et al., “Machine learning for total organic carbon analysis of environmental water samples using high-throughput colorimetric sensors”, Analyst, Vol. 145, pp. 2197-2203

### Patent Literature

PTL 1 Japanese Unexamined Patent Application Publication (Translation of PCT Application) No. 2004-514114

### SUMMARY OF INVENTION

### Technical Problem

However, when a reaction field is formed by disposing chemical reagents at a high density as in the technique of NPL 1, when a target substance to be analyzed is applied by a spray method, a chemical reagent may be eluted into a solvent on the analysis target side and may be blurred. As a result, noise and variation become large, and it is difficult to perform accurate analysis. Furthermore, in detection of a target substance, it is general to use a probe having a specific interaction that matches the target substance. It is also necessary to prepare a probe matched with an object and further to select the object to be detected in the analysis of a mixture.

In addition, as described above, PTL 1 describes an example of performing genotype identification by polymorphism analysis using particles to which a plurality of different probes are bonded, but the analysis is to determine the presence or absence of complementarity from a relative emission intensity histogram, the utilization of data including various information generated from complicated interactions and chemical reactions is insufficient, the information included in data generated from the use of a binding agent having specificity is limited, and the generation of multidimensional data or the like integrating a plurality of reaction systems is required.

Therefore, an object of the present invention is to provide an analysis system capable of detecting, as a signal, a subtle change in emission color or emission spectrum shape due to an interaction with a target substance, and acquiring a large amount of data converted from the signal in a short time and easily, furthermore, a luminescent probe that can be a molecular probe suitable for acquiring a large amount of data, or a composition for signal generation containing a carrier thereof, an ink, an analysis system using a measurement chip, a plate used therefor, and an analysis method.

### Solution to Problem

In order to achieve at least one of the above-described objects, an analysis system reflecting one aspect of the present invention is as follows.

An analysis system, comprising:
a plate comprising a plurality of reaction fields for accommodating a first component and a second component, the plurality of reaction fields being partitioned at intervals;
a signal information acquirer configured to acquire first signal information from the plate in a case where the first component is accommodated in the plurality of reaction fields, and second signal information from the plate in a case where the first component and the second component are accommodated such that the second component is further accommodated in the plurality of reaction fields from which the first signal information has been acquired; and
an analyzer for performing machine learning and analyzing a difference between the first signal information and the second signal information,
wherein
at least two of the plurality of reaction fields are regions for respectively accommodating a plurality of the first components comprising different compositions, and
at least one of the plurality of reaction fields is a region for causing a plurality of types of reactions comprising an interaction of the first component and/or the second component.

A plate reflecting one aspect of the present invention is as follows.

A plate to be used for machine learning, comprising:
a plurality of reaction fields for accommodating a first component and a second component, wherein
the plurality of reaction fields are partitioned at intervals,
at least two of the plurality of reaction fields are regions for respectively accommodating a plurality of the first components comprising different compositions, and
at least one of the plurality of reaction fields is a region for causing a plurality of types of reactions comprising an interaction of the first component and/or the second component.

An analysis method reflecting one aspect of the present invention is as follows.
disposing a first component in each of a plurality of reaction fields of a plate, the plate comprising the plurality of reaction fields for accommodating the first component and a second component, the plurality of reaction fields being partitioned from each other at intervals;
acquiring first signal information from the plate in which the first component is disposed;
further disposing the second component in each of the plurality of reaction fields of the plate from which the first signal information has been acquired;
acquiring second signal information from the plate in which the first component and the second component are disposed; and
machine learning and analyzing a difference between the first signal information and the second signal information,
wherein
in the disposing the first component, a plurality of the first components comprising different compositions are disposed in at least two of the plurality of reaction fields, and
in disposing the second component, a plurality of types of reactions comprising an interaction of the first component and/or the second component are caused in at least one of the plurality of reaction fields.

### Advantageous Effects of Invention

According to an analysis system, a plate, and an analysis method according to an embodiment of the present invention, a target substance can be accurately analyzed.

### BRIEF DESCRIPTION OF DRAWINGS

FIG. 1 is a flowchart of an analysis method according to an embodiment of the present invention;

FIG. 2 is a diagram for explaining a luminescent probe that can be used in the analysis method of the present invention;

FIG. 3A to FIG. 3D is a diagram for explaining a mechanism by which a luminescent probe used in an embodiment of the present invention exhibits its effect;

FIG. 4A and FIG. 4B are results of main component analysis in Example 1;

FIG. 5A and FIG. 5B are results of main component analysis in Example 1;

FIG. 6 illustrates the results of main component analysis in Example 2;

FIG. 7 is a cross-sectional view illustrating an example of a microarray device used in Example 3;

FIG. 8 is a partial enlarged view of a bitmap pattern of a microarray device used in Example 3;

FIG. 9 is an overall view of a bitmap pattern of a microarray device used in Example 3;

FIG. 10 is a graph illustrating the production district discrimination rate for each number of microdot light emitting parts used in the microarray device used in Example 3;

FIG. 11A illustrates linear discriminant analysis model plots when analyzing seven types and ninety five brands of beverages using the luminescent probes 1 to 15, and FIG. 11B is a confusion matrix at this time;

FIG. 12A illustrates linear discriminant analysis model plots when analyzing seven types and 95 brands of beverages using the luminescent probes 1 and 17 to 31, and FIG. 12B illustrates a confusion matrix at this time;

FIG. 13A illustrates linear discriminant analysis model plots when analyzing 7 types and 95 brands of beverages using the light emitting probes 1 to 15 and 17 to 31, and FIG. 13B illustrates a confusion matrix at this time;

FIG. 14A illustrates linear discriminant analysis model plots when analyzing 7 types and 95 brands of beverages using the light emitting probes 1 and 32 to 45, and FIG. 14B illustrates a confusion matrix at this time;

FIG. 15A illustrates linear discriminant analysis model plots when analyzing 7 types and 95 brands of beverages using the light emitting probes 46 to 60, and FIG. 15B illustrates a confusion matrix at this time; and

FIG. 16A illustrates linear discriminant analysis model plots when explanatory variables at the time of creating a discriminant model are replaced and seven types and 95 brands of beverages are analyzed, and FIG. 16B illustrates a confusion matrix at this time.

### DESCRIPTION OF EMBODIMENTS

Hereinafter, the present invention will be described in detail with reference to an embodiment. However, the present invention is not limited to these embodiments.

In addition, in the present specification, “acting nonspecifically” means that a analysis substance such as a luminescent probe described below can act not only on one type of substance but also on a plurality of substances, or can act not only on one position of a specific substance but also on a plurality of positions thereof. For example, in the embodiment described below, the plurality of types of reactions are preferably interactions that occur non-specifically between a first component described below and a second component described below.

### 1. Analysis Method

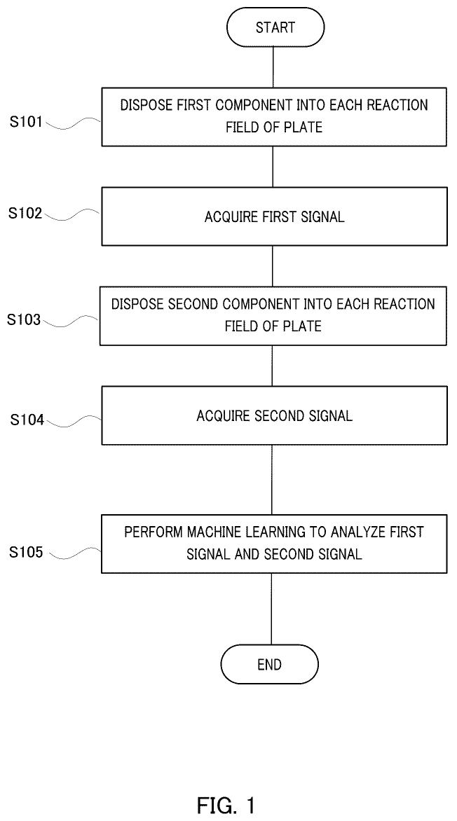

In the analysis method of the present embodiment, the analysis is performed by using a plate having a plurality of reaction fields for allowing the first component and the second component to interact with each other, the plurality of reaction fields being partitioned at intervals. A flowchart illustrating each step of the analysis method of the present embodiment is illustrated in FIG. 1. In the analysis method, the first components are disposed in a plurality of reaction fields of the above-mentioned plate (S101, hereinafter, also referred to as “first component disposing step”). Subsequently, first signal information is acquired from the plate on which the first components are disposed (S102, hereinafter, also referred to as a “first signal acquiring step”). Thereafter, the second component is disposed in each of the plurality of reaction fields of the plate (S103, hereinafter also referred to as “second component disposing step”). Then, second signal information is acquired from the plate on which the first components and the second components are disposed (S104, hereinafter also referred to as a “second signal acquiring step”). Thereafter, the difference between the first signal information and the second signal information is subjected to machine learning and analyzed by the analysis section (S105, hereinafter also referred to as “analysis step”).

The first component and the second component used in the present embodiment may be components capable of interacting with each other. However, it is preferable that one of the first component and the second component contains a target substance that is an analysis target, and the other contains a substance capable of interacting with the target substance (hereinafter also referred to as an “analysis substance (i.e., substance for analysis)”). In the present embodiment, the first component contains a analysis substance, and the second component contains a target substance, but these may be reversed.

Furthermore, although the first component and the second component may each contain an analysis substance or a target substance alone, the first component and the second component may further contain various substances and the like, such as a solvent and an impurity. Note that the “first component” referred to in the present specification does not refer to a specific compound or a specific composition, but is a general term for various compounds or various compositions to be disposed in the reaction field in the first component disposing step. Similarly, “second component” does not refer to a specific compound or a specific composition, but is a general term for various compounds or various compositions to be disposed in the reaction field in the second component disposing step.

The type of the target substance to be analyzed by the analysis method of the present embodiment is not particularly limited, and for example, the target substance may be a substance whose structure is known or a substance whose structure is unknown. Furthermore, it may be a mixture or the like of various compounds, and may be a substance, a compound, or a composition belonging to any field such as the medical field, the industrial field, the food field, or the like. Examples of the target substance belonging to the medical field include proteins, antibodies, beads with antibodies, tumor markers, and the like. On the other hand, examples of the target substance belonging to the industrial field include metal nanoparticles, carbon nanotubes, magnetic fluid, nanosilica, crystalline zirconia and the like. Examples of the target substance belonging to the food field include agricultural products and processed products thereof.

On the other hand, the type of the analysis substance is appropriately selected according to the method of acquiring signals in the first signal information acquiring step and the second signal information acquiring step. The type of the analysis substance may be any substance that, due to an interaction with the target substance, causes the second signal information acquired in the second signal information acquiring step to differ from the first signal information acquired in the first signal acquiring step. The analysis substance may be an organic compound, or an inorganic compound. However, an organic compound is more preferable from the viewpoint that signal information is easily changed when interacting with a target substance. Hereinafter, a case where a “luminescent probe” that emits light when irradiated with predetermined light is included as an analysis substance will be described as an example, but the analysis substance is not limited to the luminescent probe.

Examples of the luminescent probe include compounds having a molecular weight of 10000 or less and capable of interacting with the target substance. The molecular weight of the luminescent probe is more preferably 100 or more and 10000 or less. When the molecular weight of the luminescent probe is 10000 or less, even when the target substance has a bulky structure, the luminescent probe tends to enter the inside of the target substance and tends to interact with the target substance. Furthermore, when the luminescent probe is a compound having a relatively low molecular weight, the specificity between the luminescent probe and the target substance is low, and the luminescent probe tends to interact with a plurality of positions or a plurality of structures of the target substance. As a result, the second signal information is more likely to greatly change from the first signal information.

Examples of the luminescent probe include general phosphors. Examples of general fluorophores include fluoresceinisothiocyanate (FITC), derivatives of rhodanine (TRITC), coumarins, cyanines, CF dyes, FluoProbes, DyLight Fluors, Oyester (dye), Atto dye, HiLyte Fluors, and Alexa Fluors.

These fluorophores may also be quantum dots, proteins (e.g., green fluorescent protein (GFP)), or small molecule dyes. Examples of such small molecule dyes include xanthene derivatives (fluoresceins, rhodamines, Oregon green, eosine, texas red, etc)), cyanine derivatives (cyanines, indocarbocyanines, oxacarbocyanines, thiacarbocyanines, merocyanines, etc), naphthalene derivatives (dansyl and prodan derivatives), coumarin derivatives, oxadiazole derivatives (pyridyloxazole), nitrobenzoxadiazole, benzoxadiazole, etc), pyrene derivatives (cascade blue, etc), BODIPY (Invitrogen), oxazine derivatives (nile red, nile blue, cresyl violet, oxazine 170, etc), acridine derivatives (proflavine, acridine orange, acridine yellow, etc), arylmethine (arylmethine) derivatives (auramine, crystal violet, malachite green, etc), CF dyes (Biotium), Alexa Fluor (Invitrogen), Atto and Tracy (Sigma), tetrapyrrole derivatives (porphine, phthalocyanines, bilirubin, etc), and others (cascade yellow, azure B, acridine orange, DAPI, Hoechst33258, lucifer yellow, piroxicam, quinine and anthraquinone (anthraqinone), squarylium, oligophenylene, etc).

Another example of a luminescent probe includes a compound having a nucleic acid structure and one or more chromophores and/or luminophores bonded to the main chain of the nucleic acid structure. The type of nucleic acid structure is not particularly limited. For example, it may have an artificial nucleic acid structure. An artificial nucleic acid structure refers to a nucleic acid structure in which a non-natural portion has been introduced into a natural nucleic acid or which has been synthesized only at the nucleotide portion. An artificial nucleic acid structure is usually a completely artificially synthesized molecule which does not occur in nature. Therefore, it has a feature that it is hardly recognized by a nuclease or the like existing in the air and is hardly decomposed. The nucleic acid structure including such an artificial nucleic acid structure can stabilize the luminescent probe and enhance the emission of light from the chromophore or the luminophore, for example. Examples of nucleic acids structures include, but are not limited to, DNA, RNA, phosphorothioate oligodeoxynucleotides, 2′-O-(2-methoxy) ethyl-modified nucleic acids, siRNA, crosslinked nucleic acids, peptide nucleic acids, and morpholino antisense nucleic acids, acyclic Threoninol Nucleic Acid (aTNA), Serinol Nucleic Acid (SNAs), Peptide Nucleic Acid (PNA), Glycol Nucleic Acid (GNA), Locked Nucleic Acid (LNA), and the like. The luminophore or chromophore may be present on at least a portion (e.g., one end portion side) of the main chain of the nucleic acid structure, and a region that does not include the luminophore or chromophore may be present on the other side.

Examples of the luminescent probe having the nucleic acid structure include those having a main chain having at least one structural unit containing a sugar structure derived from pentose or hexose and a phosphate ester bond bonded to the sugar structure, and one or more chromophores and/or luminophores. The structure or position of one or more chromophores and/or luminophores is not particularly limited, but they are preferably bound to the sugar structure. Furthermore, the labeling substance preferably has a site capable of interacting with an object.

Furthermore, in the luminescent probe, the interaction part that interacts with the target substance preferably has a nucleic acid structure capable of non-specifically interacting with the target substance. Examples of the interaction between the interaction part and the target include a hydrogen bond between nucleic acids (hybridization), a hydrogen bond with a protein amide group or an amino acid in a biological substance such as an enzyme or a tumor marker, a hydrogen bond with an acid, and an interaction due to the shape of a formed steric structure.

The structure and position of a chromophore or a luminophore included in the luminescent probe are not particularly limited. For example, it may be disposed at a position separated from the above described interaction part via any other nucleic acid structure. However, the chromophore or the luminophore is preferably disposed at a position close to the interaction part, and is preferably disposed at an end portion of the interaction part or between bases of the nucleic acid structure of the interaction part. When the fluorescence emitting part and the interaction part are close to each other, the wavelengths and intensity of fluorescence emitted by the fluorescence emitting part are more likely to change in accordance with the state of interactions between the interaction part and the target substance. Note that in the present specification, a “chromophore” refers to a structure that absorbs light having a wavelength of 300 nm or more, and a “luminophore” refers to a structure that absorbs light having a wavelength of 300 nm or more and emits the light.

Further, the number of chromophores or luminophores contained in the luminescent probe is not particularly limited, and may be one or more. In the case where the luminescent probe contains a plurality of chromophores or a plurality of luminophores, the chromophores or the luminophores can be allowed to act on each other to, for example, cause self-quenching of the luminescent probe when the luminescent probe is not interacting with an object or increase the emission intensity when the luminescent probe interacts with an target substance.

The type of the dye that can be the chromophore or the luminophore contained in the luminescent probe is not particularly limited, and examples thereof include general fluorescent dyes (e.g., cyanine dyes, merocyanine dyes, acridine dyes, coumarin dyes, ethidium dyes, flavin dyes, fused aromatic ring dyes, xanthene dyes, and the like).

In addition, examples of the chromophore or the luminophore that emits fluorescence include structures derived from fluorescein, rhodamine, boron dipyrromethene, and the like. Examples of the chromophore or luminophore that emits phosphorescence include structures derived from iridium complexes, platinum complexes, and the like. Examples of the chromophores or luminophores that emits excimer light include structures derived from pyrene, anthracene, perylene, and the like. An example of a chromophore or a luminophore that emits exciplex light includes a structure derived from pyrene-dimethylaniline or the like. Examples of the chromophore or the luminophore that emits thermally activated delayed fluorescence include structures derived from 4CzIPN, DABNA, and the like. Examples of the chromophore or the luminophore that emits excited state intramolecular proton emission include structures derived from hydroxyphenylbenzoxazole and the like. Examples of the chromophore or the luminophore that emits triplet triplet annihilation emission include structures derived from 9-,-10-diphenylanthracene, rubrene, and the like. Examples of the chromophore or the luminophore that emits twisted intramolecular charge transfer emission include structures derived from diaminoanthracene, diaminonaphthalene, and the like. Examples of the chromophore or the luminophore that emits aggregated organic luminescence include structures derived from tetraphenylethene, hexaphenylsilole and the like.

The luminescent probe may be a compound that emits, for a single excitation light, two or more types of light selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregated organic luminescence.

Note that in the luminescent probe, as described below, the main chain interacts with a target substance, and along with this, the position and the interaction state of the chromophore or the luminophore bound to the main chain change. Therefore, the sugar structure is not particularly limited as long as it is a structure capable of interacting with a target substance, but the sugar structure is preferably ribose or deoxyribose. When the target substance is natural DNA or the like, deoxyribose in which nucleic acid bases are linked to each other is a β-form in DNA. Therefore, when the target substance is natural DNA, it is preferable that 50% or more of the sugar structure to which a chromophore or a luminophore is bonded is a β-form.

On the other hand, the number of the chromophores or the luminophores included in the luminescent probe may be only one, or may be two or more, as long as the above-described plurality of types of luminescence are obtained. Furthermore, when there are two or more chromophores or luminophores, the number of types of the chromophores or luminophores may be only one, or may be two or more. In addition, when there are a plurality of chromophores or luminophores, one or more chromophores or luminophores may be bonded to all the structural units of the main chain, but a chromophore or a luminophore may not be bonded to some of the structural units.

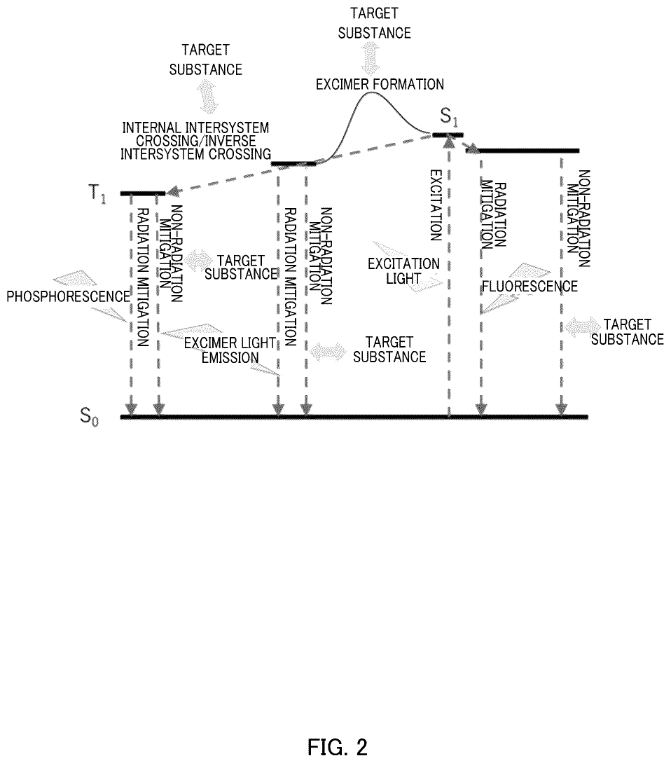

When a compound that emits a plurality of types of light in response to a single excitation light is used as a luminescent probe, complicated light emission occurs due to interaction between the target substance and the luminescent probe. For example, as illustrated in FIG. 2, when a compound that emits three different types of light, namely, fluorescence, phosphorescence, and excimer light emission, is used as the luminescent probe, the processes in which the fluorescence, the phosphorescence, and the excimer light emission are generated are changed by the interaction between the target substance and the luminescent probe, and the wavelength and the lifetime of the light are changed. That is, complicated data in which these types of light are combined is obtained according to the structure, the state, and the like of the target substance, and by analyzing this, it is possible to grasp the structure, the state, and the like of the target substance.

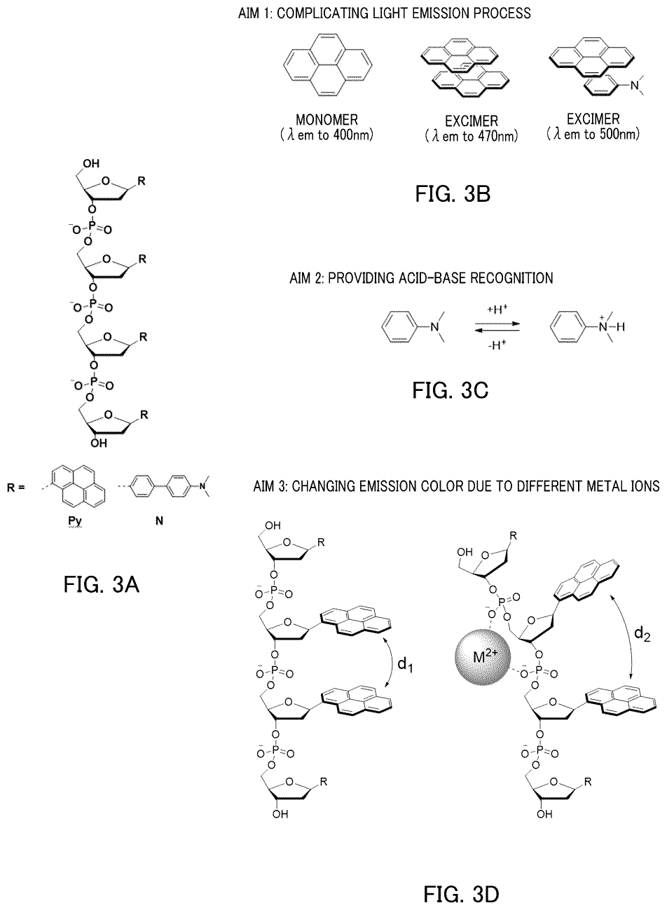

The effect expression mechanism of the luminescent probe is schematically illustrated in FIG. 3A to FIG. 3D. First, for the purpose of promoting understanding, the luminescent probe of the present embodiment is defined as a molecule having four sugar structures and four luminophores as illustrated in FIG. 3A. All of the four R's may be pyrene (Py in the drawing), dimethylaminobiphenyl (N), or a mixture thereof. One or two of the four R's may be a hydrogen atom. With such a molecular structure, a form of such a complex type fluorescent dye molecule can be constructed.

The present embodiment is characterized in that a liquid substance, a dispersion substance, or a gaseous substance serving as a target substance serving as a specimen causes a complicated interaction with a luminescent probe, and that a large amount of multidimensional data is generated as light or color signals. FIG. 3B to FIG. 3D illustrate the concept most simply and concretely, and the aim is roughly divided into three cases.

For example, assuming that all of the above-described R's are pyrene, when a specimen (target substance) is brought into contact with a plate on which the luminescent probe exists and excited with ultraviolet light, the specimen (target substance) is inserted between pyrene molecules existing in the luminescent probe. In addition, when a substance having a wide band gap that does not quench the fluorescence of pyrene (for example, an aliphatic compound or the like) is present as the target substance, the monomer luminescence of pyrene illustrated in the left diagram of FIG. 3B is observed from the luminescent probe.

When no component that interacts with pyrene is contained in the specimen (target substance), excimer light emission of pyrene illustrated in the center diagram of FIG. 3B is obtained. In addition, when a target substance (fluorescent substance) having an energy level close to the band gap of pyrene that interacts with pyrene is present in the specimen (target substance), exciplex light emission by pyrene and the fluorescent substance is obtained as illustrated in the right diagram of FIG. 3B.

Furthermore, when a metal ion such as a sodium ion or a calcium ion exists in the specimen (target substance) and the luminescent probe has the above-described phosphate group, the metal ion forms a chelate with the phosphate group existing in the main chain part of the luminescent probe. As illustrated in FIG. 3D, the intermolecular distance between pyrene molecules varies depending on the size of the metal ion. Therefore, the excimer light emission itself also changes in emission color (emission spectrum).

Furthermore, when R is dimethylaminobiphenyl (N), a change in the emission color (spectrum) of the fluorescent dye (N) occurs due to proximity of an acid and a base as illustrated in FIG. 3C. This is different from acid-base ion pairing, such as switching on/off like mineral acids (such as sulfuric acid and nitric acid) and alkali metals. In this case, since an approach distance to N continuously changes according to the acidity (easiness of providing protons) of the target substance, using this light emission phenomenon as a signal leads to expansion of a dynamic range. Further, since dimethylaminobiphenyl (N) illustrated in FIG. 3A is a Lewis base, the same interaction occurs with a Lewis acidic substance (e.g., triarylborane, trialkylaluminum, or tetraalkoxytitanium) as well as a protic acidic substance. Therefore, a specific luminescent color change occurs even for such a target substance.

Next, in the case where R's of the luminescent probe are alternately pyrene (Py) and dimethylaminobiphenyl (N), unlike the above case, there is no interaction between the specimen (target substance) and pyrene or dimethylaminobiphenyl. Therefore, exciplex light emission between pyrene and dimethylaminobiphenyl is observed, and in the case of interaction, as described above, complex light emission due to a mixed exciplex of the specimen (target substance) and pyrene and/or dimethylaminobiphenyl is obtained.

Furthermore, when one or two of the four R's on the inner side are hydrogen atoms, excimer light emission or exciplex light emission does not occur from the luminescent probe itself. Alternatively, its contribution is small. Therefore, although almost monomer emission is observed, since the steric hindrance of the hydrogen atom is small, the interaction between the target substance and R of the luminophore is enhanced, and the change in the emission signal will be enhanced.

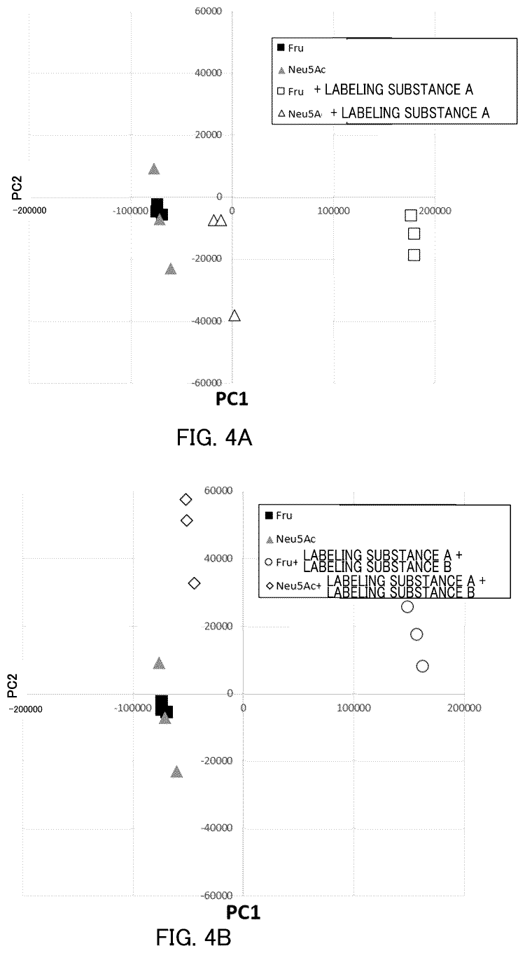

When Py or N in FIG. 4A is not a normal fluorescent substance but a phosphorescent compound or a thermally excited delayed fluorescent compound, light emission occurs at a timing delayed by several tens of nanoseconds to several microseconds from immediately after excitation, and therefore, a factor of “time” rather than emission color expands the dynamic range, and such a phenomenon is also applicable to the present embodiment in addition to the mechanisms as in FIGS. 4A and 4D described above.

Such an intermolecular interaction and a resultant subtle change in emission color or emission spectrum, a delay of emission of several microseconds, or the like, and furthermore, an extremely subtle change in a light emission phenomenon brought about by metal chelate formation due to a main chain structure have not been able to be applied as analysis information for human understanding. However, on the premise that artificial intelligence (AI) that has become generally available in recent years and machine learning and informatics utilizing the AI are used, such various light emission phenomena beyond the understanding of human beings occur, and the light emission phenomena become state description data corresponding to a target specimen (target substance). Such a new concept is a basic concept principle of the present embodiment, and is extremely useful as a new method of state description for various future research and development and production processes, furthermore, cell culture, waste liquid/wastewater/sludge treatment, and other complicated and vague specimens (target substances).

There are actual examples in which analysis using a DNA-like fluorescent compound having a similar structure has already been performed, but these are greatly different from the concept of the present embodiment in which a complex system is measured in multiple dimensions as it is and a solution is recursively obtained using AI, and it is considered that these are to be distinguished from completely different inventions.

Note that the above luminescent probe can be synthesized by the following method. First, a monomer in which the chromophore or luminophore and the phosphate ester are bonded to pentose or hexose is prepared. The monomers are polymerized into a desired sequence using a phosphoramidite method with a DNA/RNA synthesizer or the like. Such a method can synthesize a wide variety of luminescent probes depending on the type of the target substance.

Hereinafter, the analysis method of the present embodiment will be described in detail.

### (First Component Disposing Step)

In the first component disposing step, the first component is disposed in each of reaction fields of a plate including a plurality of reaction fields for allowing the first component and the second component to interact with each other.

The plate used in this step may be any plate as long as it has a plurality of reaction fields for allowing the first component and the second component to interact with each other, and the plurality of reaction fields are partitioned from each other at intervals. The plate may be in the form of a flat plate or may have recesses and projections, but is particularly preferably in the form of a flat plate. The material, size, shape, and the like of the plate are appropriately selected according to the purpose of analysis, the types of the first component and the second component, and the like.

In the plate, the positions of the reaction fields may be defined at intervals so that adjacent reaction fields are not in contact with each other. The interval is appropriately selected depending on the size of the reaction field, the types of the first component and the second component, the disposing method, and the like. For example, in a case where the first component is applied to the reaction fields by an inkjet method, a case where the first component in the form of particles is fixed, or the like, the size of each reaction field is preferably such that the diameter thereof is 100 μm or less. At this time, the interval between the adjacent reaction fields can be 200 μm or less.

In a case where the first component disposing step and the second component disposing step are performed by a machine (for example, an inkjet device or the like), a mark (formation of an uneven structure or marking) or the like indicating the position of each reaction field may not be formed on the plate. On the other hand, when a mark (formation of an uneven structure or marking) indicating the position of each reaction field is formed on the plate, it is easy to accurately dispose the first component and the second component at a desired position (reaction field) when performing the first component disposing step and the second component disposing step.

In addition, when each reaction field is formed in a concave shape or a partition wall portion is disposed around each reaction field, there is an advantage that the first component and the second component of the adjacent reaction fields are not easily mixed. In addition, for example, even in a case where the water repellent treatment portion is disposed around the reaction field, the first component and the second component of the adjacent reaction fields are less likely to be mixed. In the present embodiment, a plate in which a plurality of wells are regularly disposed is used. In such a plate including wells, the wells (reaction fields) are physically separated from each other by partition walls.

Here, the number of the reaction fields included in one plate is appropriately selected according to the type of the target substance to be analyzed, the type of the luminescent probe for labeling the target substance, and the like. The number of reaction fields may be two or more, but the greater the number, the more and multidimensional data can be acquired, and more precise analysis can be performed.

In addition, a large number of first components can be disposed by disposing respective luminescent probes in different reaction fields on a plate. Simply by adding each of the second components thereto, a large number of multidimensional data can be acquired. Preferably, the first component contains a luminescent probe, and the second component contains a target substance that is analysis target.

Here, a method of disposing the first component in each reaction field is not particularly limited, and is appropriately selected according to the type, physical properties, and the like of the first component. Examples of the method for disposing the first component include application by an inkjet device, application by a dispenser, disposing of a carrier for carrying the first component, direct fixation of the first component to the reaction field, and the like. Among these, application by an inkjet device, disposing of a carrier for supporting the first component, or direct fixation of the first component to the reaction field is particularly preferable. According to the inkjet method, it is possible to efficiently dispose the first component in a liquid state in a large number of regions and form a reaction field. This makes it possible to acquire a large amount of data. On the other hand, it is easy to independently arrange a plurality of types of first components by disposing of a particulate carrier, direct fixation, or the like. Furthermore, since the reaction fields are fixed at the time of disposing of the second component, mixing and blurring of the reaction fields due to elution can be suppressed, and the independence of the reaction fields can be maintained. In addition, in particular, in a case where the plate has a flat plate shape, there is an advantage in that it is not necessary to form a special structure such as a partition wall by using the inkjet method or fixing.

In this step, a compound or a composition having the same composition may be disposed as the first component in a plurality of reaction fields. However, first components having different compositions are disposed in two or more reaction fields among the plurality of reaction fields. That is, the first components having different compositions are disposed in different reaction fields.

The larger the number of first components having different compositions disposed in the reaction field is, the more data can be acquired, which is preferable. When the first component is a luminescent probe, it is preferable to arrange a plurality of luminescent probes in one reaction field in the first component disposing step, because a large number of multidimensional data can be obtained.

### (First Signal Information Acquiring Step)

In the first signal information acquiring step, first signal information is acquired from the plate on which the plurality of first components are disposed in the plurality of reaction fields. The first signal information acquired in this step is not particularly limited as long as it is information useful for the analysis described below. In the first signal information acquiring step, the first signal information may be acquired collectively from all the reaction fields of the plate, or the first signal information may be acquired from each of the individual reaction fields.

Examples of the first signal information include absorption spectra of ultraviolet light and visible light obtained by an ultraviolet-visible light absorption meter, fluorescence spectra and fluorescent fingerprints obtained by a fluorescent fingerprint measurement device, absorbance obtained by a circular dichroism spectrometer, a chromatogram obtained by a high-performance liquid chromatograph (HLPC), and spectral distribution changes and chromaticity changes over time when irradiated with specific excitation light. A combination of two or more of these may be used as the first signal information.

Hereinafter, these acquisition methods will be specifically described, but the method of acquiring signal information is not limited to the above-described methods.

When a fluorescent fingerprint is to be acquired, excitation light of a specific wavelength is irradiated onto a plate on which the first component is disposed, and the wavelength and intensity of the light (fluorescence or phosphorescence) emitted from the plate (first components) are measured with a spectrophotometer. Next, the waveband of the excitation light emitted by the excitation light sources is shifted by a desired range (e.g., 10 nm), and the wavelengths of the light and the intensity thereof are measured in the same manner. This is repeatedly performed, and a large amount of data is acquired. Then, the wavelength of the excitation light and the wavelength and intensity of the light emitted from the plate (first component) are converted into three dimensional data to obtain a fluorescent fingerprint. Note that in the present specification, the wavelengths and intensities of phosphorescence emitted by the plates (first components) and made into data are also referred to as “fluorescent fingerprints”. The acquisition of a fluorescent fingerprint is very useful when the above-described, and compound having a molecular weight of 10,000 or less and capable of interacting with a target substance are used as the luminescent probe. When such a luminescent probe is used, the fluorescent fingerprint is more likely to change due to interactions between the luminescent probe and the target substances, which facilitates analysis of the target substances.

On the other hand, in a case of acquiring a change in spectral distribution over time in a case of being irradiated with specific excitation light, the plate on which the first component is disposed is irradiated with excitation light of a specific wavelength for a short time. Thereafter, the spectral distribution is measured continuously or intermittently with a spectrophotometer. On the other hand, in a case of acquiring a change in chromaticity over time in a case of being irradiated with specific excitation light, the plate on which the first component is disposed is irradiated with excitation light of a specific wavelength for a short time. Thereafter, an image is acquired with a known CCD camera, CMOS camera, or the like, and the chromaticity is acquired from the acquired image. The acquisition of the spectral distribution and the chromaticity change is very useful when a compound having a main chain having at least one structural unit including the above-described a sugar structure derived from pentose or hexose and a phosphate ester bond bonded to the sugar structure, and one or more chromophores and/or luminophores bonded to the sugar structure is used as a luminescent probe. As described above, the luminescent probe emits a plurality of types of light, and the lifetime and intensity of each light emission are more likely to change due to the interaction between the luminescent probe and the target substance. Therefore, acquisition of the spectral distribution and the chromaticity change facilitates analysis of the target substance.

### (Second Component Disposing Step)

In the second component disposing step, the second component is disposed in each of the reaction fields of the plate. In the present embodiment, the second components having different compositions may be disposed in some or all of the reaction fields. On the other hand, the second components having the same composition may be disposed in all the reaction fields.

However, in the present embodiment, the combination of the first component and the second component is adjusted so that a plurality of reactions including interaction between the first component and/or the second component occur in at least one reaction field. The “plurality of reactions” may be a plurality of types of chemical bonding reactions caused by or a nonspecific reaction between the first and second components. To be specific, it may be a in which one luminescent probe non-specifically chemically binds to each of different positions in the molecules of the target substances. Alternatively, the first component or the second component (the first component in the present embodiment) may contain a plurality of luminescent probes, and these luminescent probes may be chemically bonded to different positions of the target substance.

On the other hand, the “plurality of reactions” may be a plurality of types of light-emitting reactions caused by the interaction between the first component and the second component. By utilizing a non-covalent weak interaction, i.e., a hydrogen bond, a π-π stacking interaction, a metal coordination bond, or the like, as the interaction, and changing the absorption and emission characteristics or the like of the plurality of types of light-emitting reactions, multidimensional and mass data can be generated, and more information can be obtained. The reaction may be a reaction in which a plurality of types of luminescence are generated by the interaction between the luminescent probe and the target substance.

As described above, the “plurality of types of light-emitting reactions” are preferably light-emitting reactions involving different luminescent probes, wavelengths, and types of luminescence, in order to generate a large amount of multidimensional data. Thus, complicated data in which these types of light are combined is obtained, and by analyzing this, it is possible to grasp the structure, state, and the like of the target substance.

The method for disposing the second components is not particularly limited, and is appropriately selected depending on the type and property of the second components. The method may be the same as the method for disposing the first component described above. In particular, coating with an inkjet device is preferable. According to the inkjet method, the liquid second components can be disposed in a large number of reaction fields by precise spotting for each reaction field, and mixing between the reaction fields is less likely to occur. In the second component disposing step, the second component may be disposed not only in the reaction field on the plate, but also in a region other than the reaction field, that is, a region on the plate where the first component is not disposed. On the other hand, the second component may not be disposed in a part of the reaction field where the first component is disposed.

### (Second Signal Information Acquiring Step)

In the second signal information acquiring step, second signal information is acquired from the plate on which the first component and the second component have been disposed. The second signal information acquired in this step is not particularly limited as long as it is information useful for analysis in the analysis step described below. Usually, it is preferably information acquired by the same method as the information acquired in the first signal information acquiring step. Furthermore, also in the second signal information acquiring step, the second signal information may be acquired collectively from all the reaction fields of the plate, or the second signal information may be acquired from each of the individual reaction fields.

### (Analysis Step)

In the analysis step, a difference between the first signal information acquired in the first signal information acquiring step and the second signal information acquired in the second signal information acquiring step is subjected to machine learning and analyzed. The term “machine learning” as used herein refers to learning of regularity and determination criteria from data and prediction and determination of an unknown based on the learning. The machine learning performed in this step may be supervised learning or may be unsupervised learning. Note that supervised learning refers to a learning method of learning a “relationship between an input and an output” from learning data with a correct label. Unsupervised learning refers to a learning method of learning a “structure of a data group” from learning data without a correct label, and refers to clustering, dimension reduction by main component analysis, or the like.

Specifically, data (hereinafter, also referred to as “data for analysis”) obtained by subtracting the first signal information from the second signal information is obtained, this is subjected to machine learning, and the state or the like of the target substance is analyzed. Note that the method of analyzing the analysis data in this step is appropriately selected according to the purpose, the type of the analysis data, and the like.

For example, for an ideal target substance, standard data may be prepared in advance by performing steps similar to the first component disposing step, the first signal information acquiring step, the second component disposing step, the second signal information acquiring step, and the like, and the state, structure, and the like of the target substance may be specified by comparing the standard data with the analysis data. In addition, in a case where the target substance is formed of a plurality of components, in a case where it is difficult to specify the target substance from various types of analysis (for example, quality of food or the like), or the like, standard data in a case where the target substance is in a good state and in a case where the target substance is in a bad state may be created and compared.

Note that in the case of performing the analysis, the standard data and the analysis data may be simply compared with each other, but for example, the result of the comparison between the standard data and the analysis data may be converted into a distance matrix and analyzed with a heat map (without weighting), the distance matrix may be subjected to main component analysis (weighting emphasizing anisotropy, also referred to as PCA), analysis by DL (weighting emphasizing isotropy), or the like.

On the other hand, the standard data may be a learned model or the like created in advance. The learned model can be created by, for example, a learned model generation process described below. Using the learned model, more appropriate analysis can be performed on the target substance.

In a case where the learned model is referred to, by applying the above-described analysis data to the learned model, it is possible to determine (predict) whether the target substance has a desired structure, how much the target substance includes a predetermined structure, whether the target substance is in a good state, and the like from the accumulated data or the like. Note that the prediction result may be obtained as, for example, classification, regression, clustering, abnormality detection (outlier detection), or the like.

As the learned model, for example, a plurality of prediction models are constructed based on the above-described difference between the second signal information and the first signal information (analysis data). Then, by combining results of the plurality of prediction models, information (for example, a structure, an amount, or the like) regarding the target substance may be predicted.

In a case where the structure or amount of a target substance is known in advance, for example, the prediction model can be constructed by performing machine learning in which the features of analysis data are explanatory variables and the structure, amount, or the like of the target substance is an objective variable. As the explanatory variables, numerical values representing the features of the above-described analysis data and numerical values calculated from the numerical values can be used. When the first signal information or the second signal is a fluorescent fingerprint, analysis data or the like obtained from the fluorescence intensity of the fluorescent fingerprint for each excitation wavelength can be adopted as the explanatory variable. On the other hand, the objective variable can be appropriately selected according to the purpose of analysis, and is not limited to the structure or amount of the target substance, but any other variable related to the target substance may be used.

When the learned model is created, a general analysis method (algorithm) can be applied. For the machine learning, for example, a prediction model constructed by an analysis method selected from linear regression (multiple regression analysis, partial least squares (PLS) regression, LASSO regression, Ridge regression, main component regression (PCR), and the like), random forest, decision tree, support vector machine (SVM), support vector regression (SVR), neural network, discriminant analysis, and the like can be applied.

According to the analysis method, it is possible to construct a novel analysis method for simply describing, that is, sensing a complicated state of a substance or gas serving as a specimen.

### For Another Embodiment

In the above description, the method of performing the first component disposing step, the first signal acquiring step, the second component disposing step, the second signal information acquiring step, and the analysis step has been described. However, it is also possible to omit the first signal acquiring step by forming a region where only the first component is disposed or a region where only the second component is disposed on the plate. In this case, a signal obtained from a region in which only the first component is disposed or a region in which only the second component is disposed can be handled as the first signal described above, and a signal obtained from a reaction field containing the first component and the second component can be handled as the second signal.

Further, an organic LED or the like having a microdot light emitting part may be used as the plate. In this case, RGB information or hyperspectral information may be extracted from the digital image information acquired as the first signal information and the second signal information.

### 2. Analysis System

The above-described analysis method can be executed by an analysis system including: a plate including a plurality of reaction fields for allowing a first component and a second component to interact with each other, the plurality of reaction fields being partitioned at intervals; a signal information acquisition section for acquiring first signal information from the plate when the first component is accommodated in the plurality of reaction fields and second signal information from the plate when the first component and the second component are accommodated in the plurality of reaction fields; a machine learning unit for performing machine learning of the first signal information and the second signal information; and an analysis section for performing analysis.

Note that the analysis system of the present embodiment may further include a configuration other than the plate, the signal information acquisition section, and the analysis section, and may further include an inkjet printing section for applying the first component and the second component, and the like. Hereinafter, each component of the analysis system of the present embodiment will be described. Note that the plate is the same as that described in the above-described analysis method, and a detailed description thereof will be omitted.

### (Signal Information Acquisition Section)

The signal information acquisition section is means for acquiring the first signal information and the second signal information described above.

The configuration of the signal acquisition section and the type of signal information acquired by the signal acquisition section are appropriately selected depending on the type of the target substance, the purpose of analysis, and the like. For example, the signal information acquisition section may be an ultraviolet-visible light absorption meter or a fluorescent fingerprint measurement device. Furthermore, it may be a circular dichroism spectrometer or a high-performance liquid chromatograph (HPLC), or may be a device or the like in which a predetermined excitation light source, a spectrophotometer, and an imaging section are combined. In addition, the signal information acquisition section may be a combination of two or more types thereof.

For example, the fluorescent fingerprint measurement device can be configured to include, for example, an excitation light source for emitting excitation light, a spectrophotometer for measuring the wavelength and intensity of fluorescence emitted by the first component or the mixture of the first component and the second component, and a calculation section for converting the wavelength of the excitation light, the wavelength and intensity of light emitted by the first component or the mixture of the first component and the second component into three dimensional data.

Examples of the light sources include supercontinuum light sources (broadband pulse light sources that emit intense in-phase light beams over a very wide wavelength range by using a nonlinear effect of an optical fiber, also referred to as “SC light sources”) and LEDs. With these light sources, the amount of light can be increased, and thus a fluorescent fingerprint is more likely to be clear. Note that the fluorescent fingerprint measuring device may include a plurality of light sources and a plurality of spectrophotometers.

In addition, the calculation section for converting the obtained data into three dimensional data can be a general information processing device, for example, a personal computer or the like.

An excitation light source in an device or the like in which a predetermined excitation light source is combined with a spectrophotometer or an imaging section is not particularly limited as long as it is a means capable of irradiating the plate with light having a desired wavelength for a desired time. An example of a preferable light source includes a picosecond diode laser, a tunable laser, a supercontinuum light source, and an LED light source. According to these light sources, the plate can be irradiated with light having a predetermined wavelength for a short time. In addition, the imaging section is not particularly limited as long as it is a means capable of acquiring a change over time in the light emission state of the light emission control material, and may be a known CCD camera, CMOS camera, or the like that intermittently or continuously captures an image of the plate a plurality of times.

### (Analysis Section)

The analysis section analyzes the target substance by machine learning of the difference between the first signal information and the second signal information acquired by the signal information acquisition section. To be specific, any configuration may be adopted as long as it is possible to calculate a difference between and the first signal information and the second signal information and perform various types of arithmetic processing. The analysis section may create a learned model and analyze the target substance on the basis of the learned model.

As such an analysis section, it is possible to use a general computer (general-purpose computer) including storage means such as a hard disk drive (HDD), a solid state drive (SSD), and a read only memory (ROM) for storing programs, data, and the like, and a central processing unit (CPU) for executing programs, performing calculation processing, and the like. The computer may further include input means such as a keyboard and a mouse, and output means such as a monitor and a printer.

### (Inkjet Printing Section)

The inkjet printing section may be capable of discharging the first component or the second component to a predetermined position (reaction field) on the plate. The inkjet printing section may have the same configuration as that of a general inkjet device.

### 3. Plate

The plate of the present embodiment is not particularly limited as long as the plate includes a plurality of reaction fields for allowing the first constituent and the second constituent to interact with each other, and the plurality of reaction fields are each partitioned at intervals. The structure of the plate is the same as that of the plate described in the above-described analysis method. In the plate, at least two of the plurality of reaction fields are regions for accommodating first components having different compositions. At least one of the plurality of reaction fields is a region for causing a plurality of types of reactions including interactions of the first components and/or the second components.

### 4. Effects of Present Embodiment

In the above-described analysis method and analysis system, the first component and the second component are disposed in each of the plurality of reaction fields disposed at intervals, and signal information is acquired. Therefore, the first component and the second component are not mixed with each other between the reaction fields adjacent to each other, and noise and variation are less likely to occur at the time of signal acquisition. Therefore, various information such as the detailed structure of the target substance can be obtained from the signal information generated by the interaction between the first component and the second component.

In addition, according to the above-described method and analysis system, it is also possible to obtain information on a plurality of target substances without performing separation processing or the like.

Furthermore, according to the above-described analysis method and analysis system, it is possible to collectively and easily acquire a large amount of multidimensional data. Furthermore, the present invention is a method or a system suitable for outputting data to a machine or an AI rather than a human, and can create a destructive innovation.

### EXAMPLES

### Example 1

### (1) Preparation of Target Substance Containing Liquid (Second Component)

In the present Example, D (−)-fructose (also referred to as “target substance a” or “Fru”) and N-acetylneuraminic acid (also referred to as “target substance b” or “Neu5Ac”) are used as target substances to be analyzed.

### Preparation of Target Substance Containing Liquid

D (−)-fructose (manufactured by FUJIFILM Wako Pure Chemical Industries, Ltd) was dissolved in a mixed liquid of 0.1 mol/L phosphate buffer solution pH 7.4 (manufactured by FUJIFILM Wako Pure Chemical Industries, Ltd) and dimethylsulfoxide (manufactured by Kanto Chemical Co., Inc.) (volume ratio 80:20) to prepare a subject substance-containing liquid a (20 mM fructose-containing liquid).

### Preparation of Target Substance Containing Liquid b

N-acetylneuraminic acid (manufactured by FUJIFILM Pure Chemical Industries, Ltd) was dissolved in a mixed liquid (volume ratio 80:20) of 0.1 mol/L phosphate buffer solution pH 7.4 (manufactured by FUJIFILM Pure Chemical Industries, Ltd) and dimethylsulfoxide (manufactured by Kanto Chemical Co., Inc.) to prepare a subject substance-containing liquid b (20 mM N-acetylneuraminic acid-containing liquid).

### (2) Preparation of Luminescent Probe Containing Liquid (First Component)

### Preparation of Luminescent Probe Containing Liquid A

4-(1-Phenyl-1H-benzoimidazol-2-yl) phenylboronic acid (manufactured by Tokyo Chemical Industry Co., Ltd., molecular weight 314.15, fluorescent substance) was dissolved in a mixed liquid of 0.1 mol/L phosphate buffer pH 7.4 and dimethylsulfoxide (manufactured by Kanto Chemical Co., Inc.) (volume ratio 80:20) to prepare a luminescent probe-containing liquid A (0.00266 mM 4-(1-phenyl-1H-benzoimidazol-2-yl) phenylboronic acid-containing liquid).

### Preparation of Luminescent Probe Containing Liquid B

Alizarin Red S (manufactured by FUJIFILM Wako Pure Chemical Industries, Ltd., molecular weight: 342.26, a fluorescent substance) was dissolved in a mixed liquid of 0.1 mol/L phosphate buffer solution pH 7.4 and dimethylsulfoxide (manufactured by Kanto Chemical Co., Inc.) (volume ratio 80:20) to prepare a luminescent probe-containing liquid B (0.00266 mM alizarin red S-containing liquid).

### Preparation of Luminescent Probe Containing Liquid C

4-methylesculetin (manufactured by Tokyo Chemical Industry Co., Ltd., molecular weight: 192.17, a fluorescent substance) was dissolved in a mixed liquid of 0.1 mol/L phosphate buffer solution pH 7.4 and dimethylsulfoxide (manufactured by Kanto Chemical Co., Inc.) (volume ratio 80:20) to prepare a luminescent probe-containing liquid C (0.00266 mM 4-methylesculetin-containing liquid).

### Preparation of Luminescent Probe Containing Liquid A+B

The luminescent probe-containing solutions A and B were mixed at a volume ratio of 1:1 to obtain a luminescent probe-containing solution A+B.

### Preparation of Luminescent Probe Containing Liquids A+C

The luminescent probe-containing solutions A and C were mixed at a volume ratio of 1:1 to obtain a luminescent probe-containing solution A+C.

### (3) First Component Disposing Step

A 96-well microplate in which wells with opening diameters of 7 mm were disposed at intervals of 9 mm in 12 columns and ×8 rows was prepared. On the 96-well microplate, 100 μl of each of the first components shown in Table 1 below was disposed by an automatic dispensing device (NichiMart CUBE) to form a plurality of reaction fields.

### (4) First Signal Information Acquiring Step

The 96-well microplate in which the above-described first component had been disposed was set in a SPARK multi-detection mode microplate reader (manufactured by TECAN), the excitation wavelengths were changed to 230 to 590 nm in 20 nm increments, and the intensity of fluorescence (wavelengths of 280 to 740 nm) from the plate upon irradiation with the respective excitation wavelengths was measured. Detection values in the vicinity of the excitation wavelengths±30 nm at the time of fluorescence measurement were excluded because they were greatly influenced by the leakage of the excitation light. Next, the excitation wavelength, the fluorescence wavelength, and the intensity of the fluorescence were converted into three dimensional data to create a fluorescent fingerprint. Three fluorescent fingerprints were created for each sample.

### (5) Second Component Disposing Step

As shown in Table 1 below, 100 μl of each of the second components was disposed on the 96-well microplate after the first signal information acquiring step by an automatic dispensing device (NichiMart CUBE).

### (6) Second Signal Information Acquiring Step

The 96-well microplate in which the above-described first and second components were disposed was set in a SPARK multi-detection mode microplate reader (manufactured by TECAN), the excitation wavelengths were changed to 230 to 590 nm in 20 nm increments, and the intensity of fluorescence (wavelengths of 280 to 740 nm) from the plate when each of the excitation wavelengths was applied was measured. Detection values in the vicinity of the excitation wavelengths±30 nm at the time of fluorescence measurement were excluded because they were greatly influenced by the leakage of the excitation light. Next, the excitation wavelength, the fluorescence wavelength, and the intensity of the fluorescence were converted into three dimensional data to create a fluorescent fingerprint. Three fluorescent fingerprints were created for each sample.

TABLE 1

| | First component (luminescent probe) | | | Second component (target substance) | | |
|---|---|---|---|---|---|---|
| | | | Application | | | Application |
| | | | amount | | | amount |
| | Type | Substance name | (mM) | Type | Substance name | (mM) |
| Well 1 | A | 4-(1-phenyl-1H-benzoimidazol-2-yl) | 0.001 | a | D(−)-Fructose | 5.0 |
| | | phenylboronic acid | | | | |
| Well 2 | A | 4-(1-phenyl-1H-benzoimidazol-2-yl) | 0.001 | b | n- | 5.0 |
| | | phenylboronic acid | | | Acetylneuraminic | |
| | | | | | acid | |
| Well 3 | A | 4-(1-phenyl-1H-benzoimidazol-2-yl) | 0.001 | — | — | — |
| | | phenylboronic acid | | | | |
| Well 4 | B | Alizarin red S | 0.001 | a | D(−)-Fructose | 5.0 |
| Well 5 | B | Alizarin red S | 0.001 | b | n- | 5.0 |
| | | | | | Acetylneuraminic | |
| | | | | | acid | |
| Well 6 | B | Alizarin red S | 0.001 | — | — | — |
| Well 7 | C | 4-Methylesculetin | 0.001 | a | D(−)-Fructose | 5.0 |
| Well 8 | C | 4-Methylesculetin | 0.001 | b | n- | 5.0 |
| | | | | | Acetylneuraminic | |
| | | | | | acid | |
| Well 9 | C | 4-Methylesculetin | 0.001 | — | — | — |
| Well 10 | A + B | 4-(1-phenyl-1H-benzoimidazol-2-yl) | 0.001 + | a | D(−)-Fructose | 5.0 |
| | | phenylboronic acid + Alizarin red S | 0.001 | | | |
| Well 11 | A + B | 4-(1-phenyl-1H-benzoimidazol-2-yl) | 0.001 + | b | n- | 5.0 |
| | | phenylboronic acid + Alizarin red S | 0.001 | | Acetylneuraminic | |
| | | | | | acid | |
| Well 12 | A + B | 4-(1-phenyl-1H-benzoimidazol-2-yl) | 0.001 + | — | — | — |
| | | phenylboronic acid + Alizarin red S | 0.001 | | | |
| Well 13 | A + C | 4-(1-phenyl-1H-benzoimidazol-2-yl) | 0.001 + | a | D(−)-Fructose | 5.0 |
| | | phenylboronic acid + 4- | 0.001 | | | |
| | | Methylesculetin | | | | |
| Well 14 | A + C | 4-(1-phenyl-1H-benzoimidazol-2-yl) | 0.001 + | b | n- | 5.0 |
| | | phenylboronic acid + 4- | 0.001 | | Acetylneuraminic | |
| | | Methylesculetin | | | acid | |
| Well 15 | A + C | 4-(1-phenyl-1H-benzoimidazol-2-yl) | 0.001 + | — | — | — |
| | | phenylboronic acid + 4- | 0.001 | | | |
| | | Methylesculetin | | | | |
| Well 16 | | | | a | D(−)-Fructose | — |
| | | | | | n- | |
| Well 17 | | | | b | Acetylneuraminic | — |
| | | | | | acid | |

### (7) Analysis Step

With respect to the first signal information acquired in the first signal information acquiring step and the second signal information acquired in the second signal information acquiring step, data frames each having a value of fluorescence wavelength-excitation wavelength of the fluorescent fingerprint as a variable were prepared. Then, the data frame of the second signal information was calibrated using the data frame of the first signal information as a blank. The calibrated data frame was converted into a distance matrix.

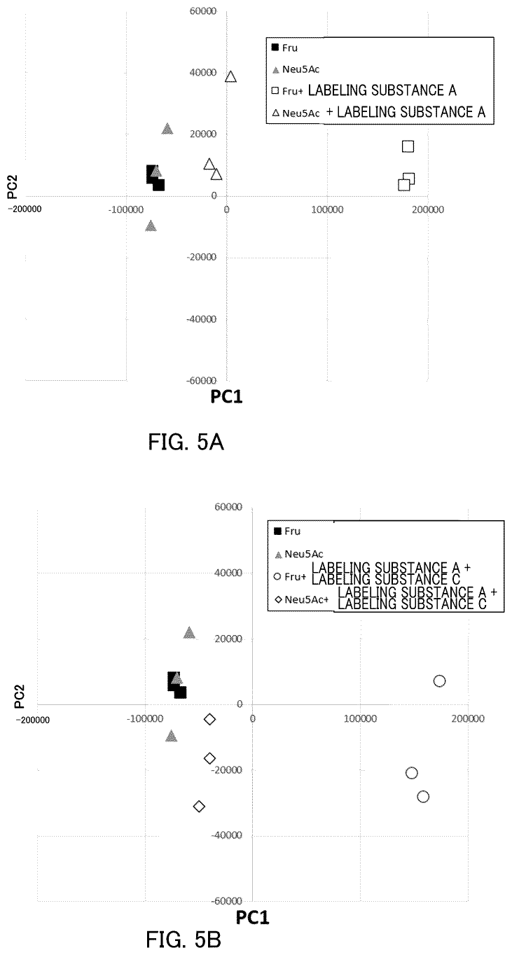

Principal component analysis (machine learning) was performed on the data frame calibrated above. Next, dot plots were drawn with the main component 1 (PC1) and the main component 2 (PC2) as axes. The results of the main component analysis are illustrated in FIG. 4A, FIG. 4B, FIG. 5A, and FIG. 5B. Principal component analysis was performed on wells 16, 17, 1, and 2 of Table 1 (FIG. 4A and FIG. 5A), wells 16, 17, 10, and 11 of Table 1 above (FIG. 4B), and wells 16, 17, 13, and 14 of Table 1 above (FIG. 5B). The graph represents the variation of data in the data space, and data points plotted nearby indicate similar changes in fluorescent fingerprints. On the other hand, data points plotted far from each other indicate a relatively large change in the fluorescent fingerprint.

### Results

As illustrated in FIGS. 4A and 4B, a change in fluorescent fingerprint was observed in the luminescent probe A. Furthermore, it is found that the distances of the values of the main components (distances of the values of 4A and 4B) are farther in the case of combining the luminescent probes A and B (Fig. PC1) than in the case of using the luminescent probe A alone (Fig. PC2). Similarly, as illustrated in FIG. 5A and FIG. 5B, a change is also observed in the case of using only the luminescent probe A (FIG. 5A), and it is found that the distances of the values of the main components (distances of the values of PC1 and PC2) are larger in the case of combining the luminescent probes A and C (FIG. 5B).

### Example 2

### (1) Synthesis of First Component (Luminescent Probe)

All reactions were performed under a nitrogen atmosphere in oven-dried glassware unless otherwise noted. All chemical products were purchased from Aldrich or TCI or Kanto Chemical and used as is without further purification.

### [Synthesis of Monomer 1]

Based on the following reaction formula, a monomer 1 having a main chain containing a phosphate ester and a luminophore bonded to the main chain was synthesized via intermediates 1 to 6.

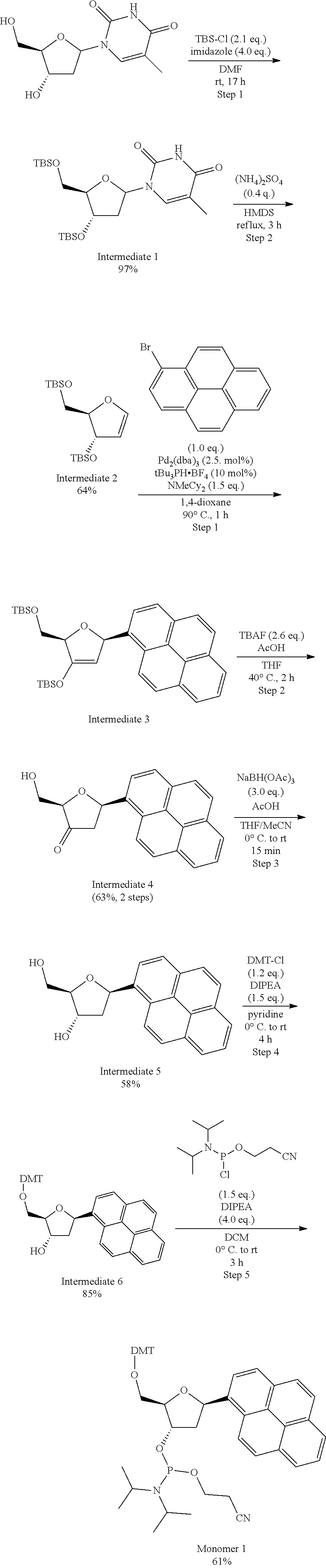

### Synthesis of Intermediate 1

Thymidine (15.0 g, 61.9 mmol) and imidazole (16.9 g, 248 mmol) were dissolved in DMF (124 mL), tert-butyldimethylsilylchloride (19.6 g, 130 mmol) was added, and the mixture was stirred at room temperature for 17 hr. To the reaction mixture was added water, and the mixture was partitioned and extracted with ethyl acetate. The obtained organic phase was dried over magnesium sulphate, and the solvents were distilled off to obtain the desired intermediate 1 as a colorless solid (28.3 g, 97%).

### Synthesis of Intermediate 2

Intermediate 1 (28.3 g, 60.1 mmol) and ammonium sulphate (12.7 g, 96.2 mmol) were dissolved in hexamethyldisilazane (314 mL, 1.50 mol), and the mixture was heated under reflux for 3 hours. The resulting crude product was purified by silica gel column chromatography to obtain the desired intermediate 2 as a brown liquid (13.2 g, 64%).

### Synthesis of Intermediate 4

A mixture of intermediate 2 (10.1 g, 29.3 mmol), 1-bromopyrene (8.24 g, 29.3 mmol), tris (dibenzylideneacetone) dipalladium (0) (671 mg, 733 μmol), tri-tertbutylphosphonium tetrafluoroborate (850 mg, 2.93 mmol), dicyclohexylmethylamine (9.35 mL, 44.0 mmol), 1,4-dioxane (100 mL) was heated at 90° C. for 1 hour. Water was added to stop the reaction, and liquid separation and extraction were performed with ethyl acetate. The obtained organic phase was dried over magnesium sulfate, and the solvent was distilled off to give a crude product containing intermediate 3, which was used as is in the next reaction.

To the crude product containing intermediate 3 were added TFL 100 mL, 1 M tetrabutylammonium fluoride in THF (117 mL, 117 mmol), and ethyl acetate (6.74 mL, 117 mmol), and the mixture was stirred at 40° C. for 2 hr. Water was added to stop the reaction, and liquid separation and extraction were performed with ethyl acetate. The obtained crude product was purified by silica gel column chromatography to obtain the desired intermediate 4 as a light brown solid (5.87 g, 63%).

### Synthesis of Intermediate 5

Solutions of sodium triacetylborate (11.8 g, 55.8 mmol) and acetic acids (7.87 mL, 138 mmol) in acetonitrile 93 mL were cooled to 0° C., and solutions of the intermediates 4 (5.87 g, 18.6 mmol) in THF (62 mL) were added dropwise. After completion of the dropwise addition, the mixture was warmed to room temperature and stirred for 15 min, and water was added to stop the reaction. Liquid separation and extraction were performed with ethyl acetate, the obtained organic phase was dried over magnesium sulfate, and the solvent was distilled off to obtain a crude product. Purification by silica gel column chromatography and reversed-phase HPLC afforded the desired intermediate 5 as a colorless solid (3.44 g, 58%).

### Synthesis of Intermediate 6

A mixture of intermediate 5 (3.44 g, 10.8 mmol), 4-,-4′-dimethoxytritylchloride (4.40 g, 13.0 mmol), ethyldiisopropylamine (2.82 mL, 16.2 mmol) and dehydrated pyridine (54 mL) was stirred at room temperature for 4 hours, and then methanol was added to stop the reaction. The solvents were distilled off and the obtained crude product was purified by silica gel column chromatography to obtain the desired intermediate 6 as a colorless viscous solid (5.71 g, 85%).

### Synthesis of Monomer 1

To a mixture of intermediate 6 (5.71 g, 9.20 mmol), ethyldiisopropylamine (6.42 mL, 36.8 mmol), and dehydrated dichloromethane (92 mL) was added dropwise 2-cyanoethyldiisopropylchlorophosphoroamidite (3.08 mL, 13.8 mmol) at 0° C. After heating to room temperature and stirring for 3 hours, the solvent was distilled off to obtain a crude product. The residue was purified by silica gel column chromatography to obtain a target luminescent probe monomer 1 as a colorless solid (4.64 g, 61%).

### [Preparation of Monomer 2]

Monomer 2, a reagent of the structure illustrated below, was purchased from Glen Research (Sterling, Va).

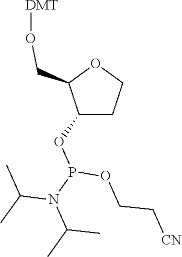

### [Synthesis of Light Emitting Probes 1 to 16 (First Components 1 to 16)]

According to a conventional method, as illustrated in Table 2 below, 16 types of oligonucleotides (Seg1 to 16) having a mixed sequence of the monomer 1 and the monomer 2 were synthesized. DNA synthesis reagents were purchased from Glen Research (Sterling, Va). In addition, all of the oligonucleotides were synthesized with a DNA/RNA synthesizer NTS T-series manufactured by Nippon Technoservice Co., Ltd. using a standard protocol for a phosphoramidites based coupling procedure. Each oligonucleotide solid-phase support obtained by the automatic synthesis was reacted with ammonium water at room temperature for 2 hours and cut out from the solid phase, and the solvent was dried and solidified with a centrifugal drying device, and then ultrapure water was added to obtain first components 1 to 16 containing luminescent probes 1 to 16, respectively. It was confirmed that the luminescent probes 1 to 16 emit fluorescence and excimer by specific excitation light (for example, light of wavelengths 350 nm).

TABLE 2

| | | Luminescent probe | | | | | | | | | | | | | | | |
|---|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 | 16 |
| Monomer | 5′ | 1 | 1 | 1 | 1 | 1 | 1 | 1 | 1 | 2 | 2 | 2 | 2 | 2 | 2 | 2 | 2 |
| sequence | 3′ | 1 | 1 | 1 | 1 | 2 | 2 | 2 | 2 | 1 | 1 | 1 | 1 | 2 | 2 | 2 | 2 |
| | | 1 | 1 | 2 | 2 | 1 | 1 | 2 | 2 | 1 | 1 | 2 | 2 | 1 | 1 | 2 | 2 |
| | | 1 | 2 | 1 | 2 | 1 | 2 | 1 | 2 | 1 | 2 | 1 | 2 | 1 | 2 | 1 | 2 |

| | | | | | | | | | | | | | | | | |
|---|---|---|---|---|---|---|---|---|---|---|---|---|---|---|---|---|
| Number of | 4 | 4 | 4 | 2 | 3 | 2 | 2 | 1 | 3 | 2 | 2 | 1 | 1 | 1 | 1 | 0 |
| chromophores or | | | | | | | | | | | | | | | | |
| luminophores | | | | | | | | | | | | | | | | |

### (2) First Component Disposing Step

A 96-well microplate in which wells with opening diameters of 7 mm were disposed at intervals of 9 mm in 12 columns and ×8 rows was prepared. In the 96-well microplate, 100 μl of each of the luminescent probes 1 to 16 was disposed by an automatic dispensing device (NichiMart CUBE, manufactured by NICHIRYO) to form a plurality of reaction fields.

### (3) First Signal Information Acquiring Step

The 96-well microplate in which the above-described first component was disposed was irradiated with excitation light (wavelength 350 nm), and the fluorescence spectrum was obtained as the first signal information.

### (4) Second Component Disposing Step

On the 96-well microplate after the first signal information acquiring step, 100 μl of each of the three types of soft drinks (second components 1 to 3) was disposed by an automatic dispenser (NichiMart CUBE, manufactured by NICHIRYO Co., Ltd).

### (5) Second Signal Information Acquiring Step

The 96-well microplate in which the above-described first component and second component were disposed was irradiated with excitation light (wavelength 350 nm), and the fluorescence spectrum was obtained as the second signal information.

### (6) Analysis Step

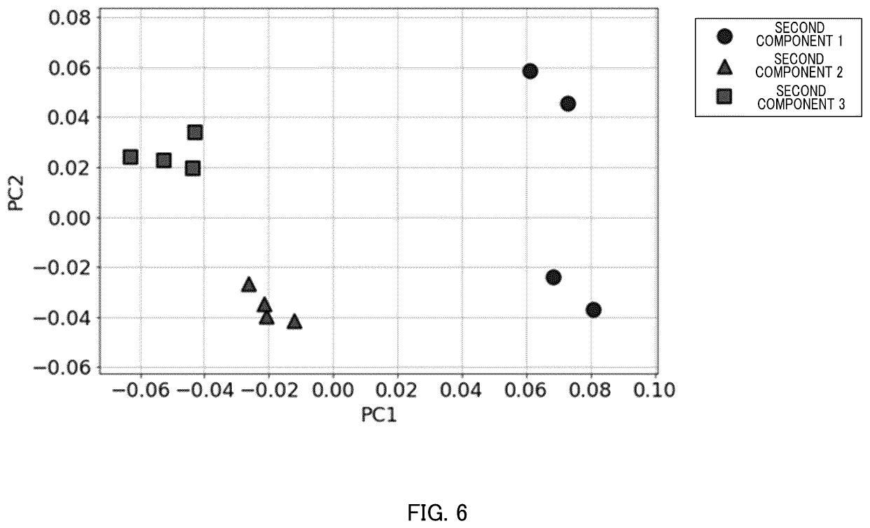

Analysis data was calculated by subtracting the first signal information acquired in the first signal information acquiring step from the second signal information acquired in the second signal information acquiring step. When main component analysis (machine learning) was performed using the analysis data as explanatory variables, it was possible to separate the second components by type in the two-dimensional space in which main components 1 and 2 were plotted, as illustrated in FIG. 6.

### Example 3

### (1) Production of Microarray Devices (Plates)

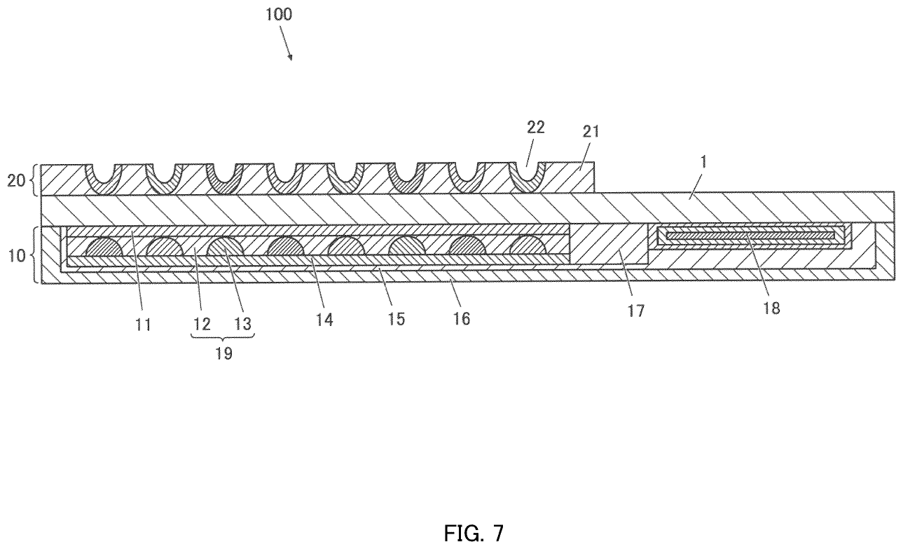

Microwells were produced on an OLED substrate including microdot light emitting parts by the following method, to produce a microarray device (100) including a plateau unit (10) with the structure illustrated in FIG. 7.

### Production of OLED Substrate Including Microdot Light Emitting Part

### <Production of Anode Member>

### (Production of Support)

First, A gas barrier layer was formed on the entire surface of one side of a polyethylene naphthalate film (manufactured by Teijin DuPont, hereinafter abbreviated as “PEN film”). For the formation of the gas barrier layer, an atmospheric-pressure plasma discharge treatment device having the configuration described in Japanese Unexamined Patent Publication No. 2004-68143 was used. The material of the gas barrier layer was silicone oxide (SiOₓ1<X≤4). The thickness of the gas barrier layer was 500 nm. Thus, a flexible support (1) having gas barrier properties with an oxygen transmission rate of 0.001 mL/(m²·24 h) or less and a water vapor transmission rate of 0.001 g/(m²·24 h) or less was produced.

### (Formation of Anode)

On the gas barrier layer (not illustrated) of the support (1), an ITO (indium-tin-oxide) film having 30 mm×30 mm areas and 120 nm thicknesses was formed by a sputtering method. Then, patterning was performed by a photolithography method to form the anodes (11) of the side 20 mm×10 mm and extraction electrodes (not illustrated). Next, using an atmospheric pressure plasma discharge treatment device, the support (1) on which the anode (11) had been formed was washed and subjected to lyophilic treatment. Argon gas was used as a discharge gas, and oxygen gas was used as a reactive gas, which were supplied at 25° C. and 1 L/(min·cm). The power source used for plasma generation was PH-F2-K manufactured by Heiden Laboratory Co., Ltd, and a voltage of about 2 kV was applied to generate plasma.

### (Formation of Receiving Layer Insulating Part)

Next, ink 1 for forming an insulating part having the following composition was ejected by an inkjet method so as to cover the entire positive electrode surface under the condition that the layer thickness after drying was a 100 nm. Next, the ink 2 for forming an insulating part was injected under the condition that the layer thickness after drying was 20 nm. For ejection of the inks 1 and 2, a piezoelectric inkjet printer head “KM1024i” manufactured by Konica Minolta, Inc. was used. Thus, the insulating part (12) of the receiving layer (19) was formed.

### <Ink 1 for Forming Insulating Part>

o-Xylene: 800 parts by mass
Tetralin: 200 parts by mass

The following compound (weight average molecular weight Mw=40000): 30 parts by mass

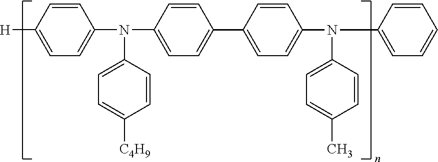

### <Ink 2 for Forming Insulating Part>

Syndiotactic polystyrene (weight-average molecular weight Mw=280000): 10 parts by mass

Propylene glycol monomethyl ether acetate: 1000 parts by mass

### (Formation of Microdot Light Emitting Parts (13) 1 to 9)

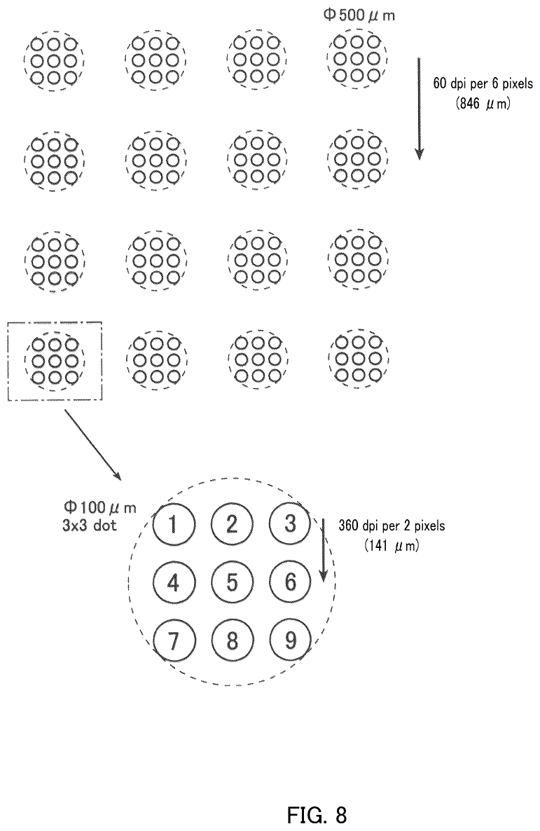

Inks 1 to 9 for forming a light emitting part having the following compositions were ejected onto the insulating part (12) of the receiving layer (19) using the inkjet printer head “KM1024i” in the same manner as described above. The bitmap pattern illustrated in FIG. 8 was used as the input data. Specifically, microdot light emitting parts (13) 1 to 9 were formed within circles corresponding to the positions of the microwell structures (22) of 60 dpi, 6 pixels each, and 500 μm in diameter to be produced below. Within the circle, microdot light emitting parts 1 to 9 were formed in a 3×3 array with a diameter of 100 μm and a resolution of 360 dpi and every 2 pixels. In FIG. 8, a broken line circle is a circle corresponding to the production position of the microwell structure part (22). In FIG. 8, the solid-line circles are the microdot light emitting parts (13). The inks 1 to 9 for forming a light emitting part contain one or more of the following luminescent dopants (luminescent compounds) Dp-1 to Dp-9, respectively. In each of the microdot light emitting parts (13), the insulating part (12) is once dissolved by the ejected ink, and the light emitting dopant and the host compound in the light emitting part-forming ink are mixed and dried again, thereby forming the microdot light emitting part (13). The ink was ejected onto the insulating part (12) under conditions such that the layer thickness of the thus formed microdot light emitting parts (13) was 30 nm. Next, the resultant was dried under nitrogen at 120° C. for 30 minutes, thereby forming microdot light emitting parts (13).

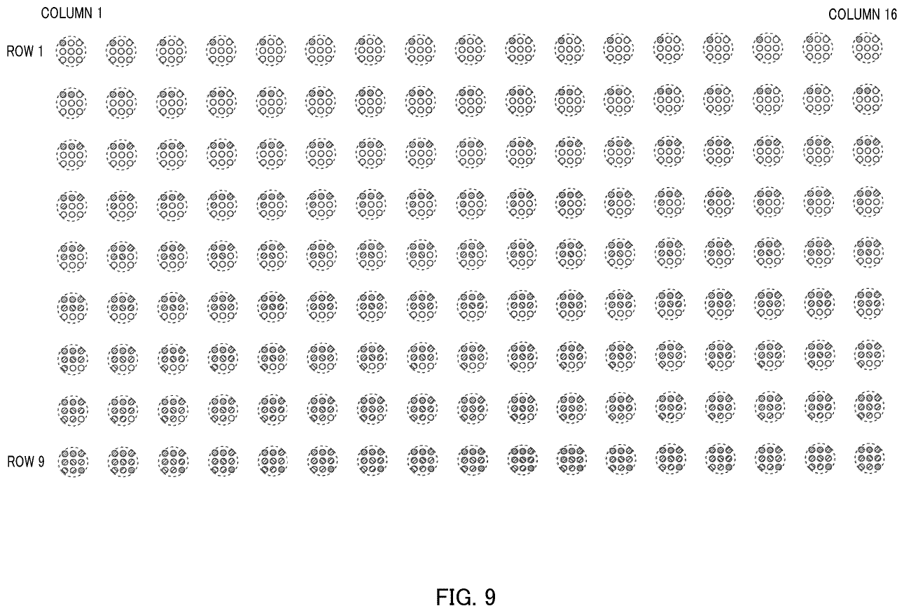

When the microdot light emitting part (13) was formed, each of the light emitting part forming inks 1 to 9 was applied to a position corresponding to the microwell structure part (22) in a pattern illustrated in FIG. 9 and Table 3 to produce the microdot light emitting part (13).

### <Coating Liquid for Forming Light Emitting Part>

Host compound (KH-1): 22.8 parts by mass
Luminescent dopant compound: 7.2 parts by mass
Propylene glycol monomethyl ether acetate: 1000 parts by mass

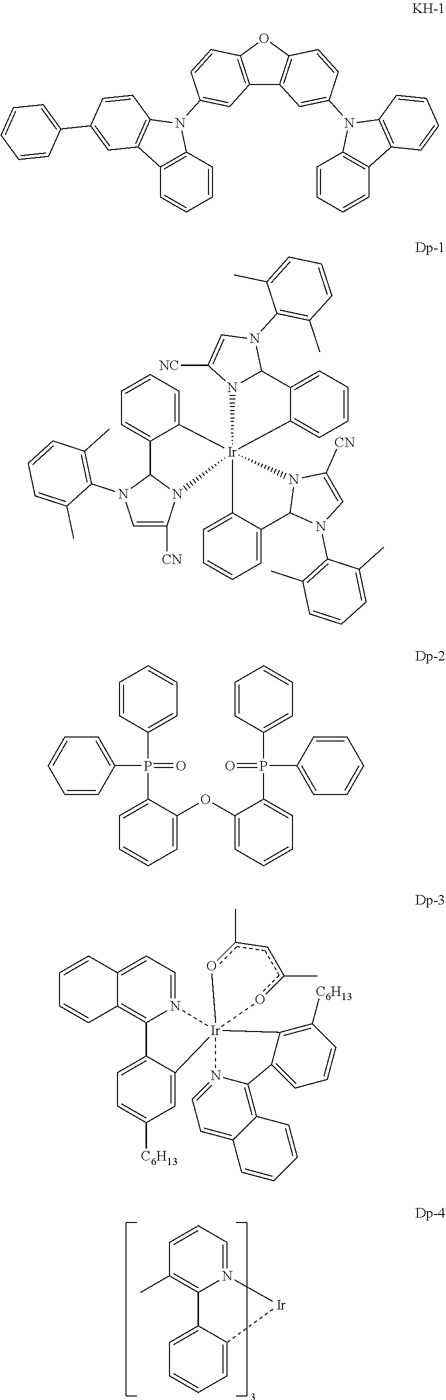

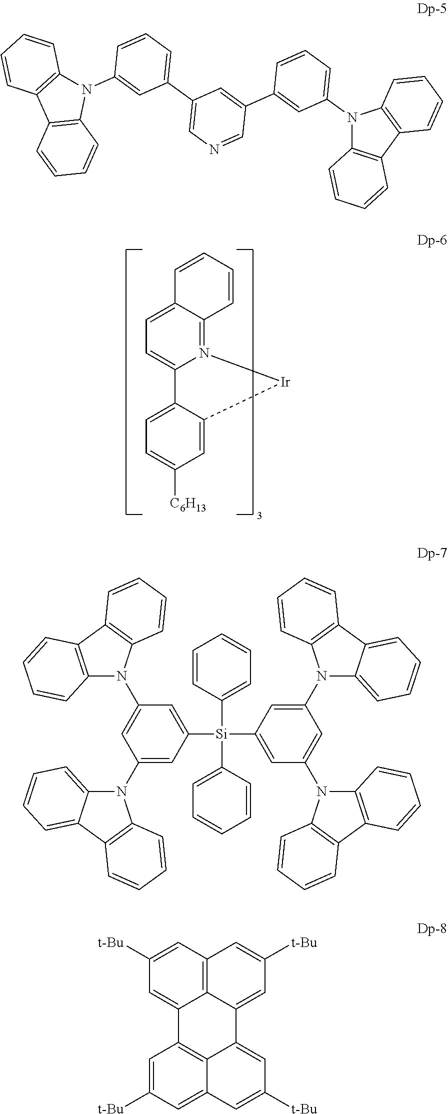

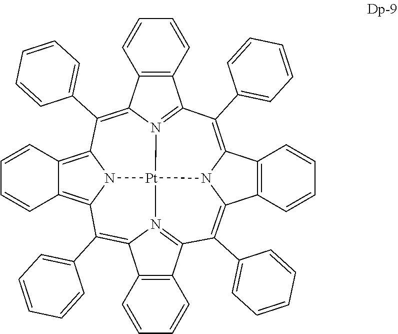

The maximum emission wavelengths of the luminescent dopants (luminescent compounds) Dp-1 to Dp-9 are as follows.
Dp-1: 477 nm, blue light region
Dp-2: 31 nm, ultraviolet region
Dp-3: 612 nm, green-red light region
Dp-4: 522 nm, green-red light region
Dp-5: 347 nm, ultraviolet region
Dp-6:583 nm, green-red light region
Dp-7: 362 nm, ultraviolet region
Dp-8: 487 nm, blue light region
Dp-9: 765 nm, green-red light region

TABLE 3

| | Microdot light emitting part | | | | | | | | |
|---|---|---|---|---|---|---|---|---|---|
| | 1 | 2 | 3 | 4 | 5 | 6 | 7 | 8 | 9 |
| First row | Yes | No | No | No | No | No | No | No | No |
| Second row | Yes | Yes | No | No | No | No | No | No | No |
| Third row | Yes | Yes | Yes | No | No | No | No | No | No |
| Fourth row | Yes | Yes | Yes | Yes | No | No | No | No | No |
| Fifth row | Yes | Yes | Yes | Yes | No | No | No | No | No |
| Sixth row | Yes | Yes | Yes | Yes | Yes | No | No | No | No |
| Seventh row | Yes | Yes | Yes | Yes | Yes | Yes | No | No | No |
| Eighth row | Yes | Yes | Yes | Yes | Yes | Yes | Yes | No | No |
| Ninth row | Yes | Yes | Yes | Yes | Yes | Yes | Yes | Yes | No |

### (Connection of Circuit and Power Receiving Antenna)

An extraction anode (not illustrated) and an extraction cathode (not illustrated) were connected to the anode pad and the cathode pad of an on-board NCF tag IC: NTAG213F (manufactured by NXP) (17), respectively, to form an anode member. The on-board NCF tag IC: NTAG213F (17) is a power receiving antenna that serves as the power receiving section (18).

### <Production of Cathode Member>

### (Production of Cathode Film)

A separately prepared PEN film was attached to the vacuum vapor deposition device. Furthermore, a resistance-heated boat made of tungsten filled with silver was attached to the vacuum vapor deposition device, and the pressure in the vacuum tank was reduced to 4×10⁻⁵ Pa. Thereafter, the boat was heated by applying an electric current, and silver was vapor-deposited to form a 100 nm cathode (14). The silver surface of the cathode film taken out from the vacuum vapor deposition device was spin-coated with the following ink for forming an electron injection adhesive layer in 500 rpm. Next, the coating film was dried by 120° C. for 10 min on a hotplate. Next, the resultant was cut into a size capable of covering the anode (11) and being connected to an extraction electrode to obtain a cathode film.

### (Ink for Forming Electron Injection Adhesive Layer)

PFN-Br (manufactured by Lumtec, molecular weight: 10000): 20 parts by mass
Branched polyethyleneimine (manufactured by Aldrich, molecular weight: 10,000): 20 parts by mass
2-Propanol: 1000 parts by mass

### (Production of Sealing Laminated Member)

A flexible base material (16) having gas barrier properties was separately prepared. On the substrate, a thermosetting adhesive shown below was uniformly applied as a sealing adhesive to a thickness of 20 μm along the barrier surface of the substrate using a dispenser. This was dried under vacuum at 100 Pa or below for 12 hours. Furthermore, the sealing member (15) was moved to a nitrogen gas atmosphere having a dew-point temperature of −80° C. or lower and an oxygen content of 0.8 ppm, and dried for 12 hours or more. The water content of the sealing adhesive was adjusted to 100 ppm or less.

An epoxy-based adhesive obtained by mixing the following (A) to (C) was used as the thermosetting adhesive.
(A) Bisphenol A diglycidyl ether (DGEBA)
(B) Dicyandiamide (DICY)
(C) Epoxy adduct-based curing accelerator

The above-described cathode film was disposed on the adhesive layer (sealing member (15))) so that the cathode (14) was exposed, to thereby obtain a sealing laminated member.

The cathode (14) of the sealing laminated member, and the receiving layer surface including the light emitting part (13) of the anode member and the extraction electrode on which the NFC tag (17) was disposed were disposed and brought into close contact with each other. Next, the resultant was adhered and sealed under pressure-bonding conditions of a temperature of 90° C. and a pressure of 0.1 MPa using a vacuum laminator, to obtain an OLED substrate.

### Formation of Microwell Structure Part

An ink for forming a microwell structure part having the following composition was ejected at a head heating temperature of 50° C. onto the surface of the substrate (1) where the anode (11) and the like were not formed. As input data, bitmap patterns as illustrated in FIGS. 8 and 9 were used. In accordance with these bitmap patterns, the outside of a 500-μm-diameter circle was solidly coated every 6 pixel so that the inside of the circle was blank. The ejection was performed twice by changing the inkjet printing sweeping direction by 90°. The film was cured by irradiation with a UV-LED of an illumination 2000 mW/cm² for 10 seconds, and the thickness of the cured film at a flat portion was defined as 10 μm. The hole part surrounded by the cured composition was defined as the microwell structure part (22). In the multi-well unit (20), the layer of the cured composition is the cured layer (21).

### <Ink for Forming Microwell Structure Part>

Diphenyl (2,4,6-trimethylbenzoyl) phosphineoxide (manufactured by Sigma—Aldrich): 2 parts by mass
2-Phenoxyethyl acrylate (manufactured by TCI): 45 parts by mass
Phenoxydiethylene glycol acrylate (manufactured by Shin—Nakamura Chemical Co., Ltd): 45 parts by mass
Polyacrylic acid (manufactured by Sigma—Aldrich, molecular weight of 450000): 8 parts by mass

### (2) Disposing of Luminescent Probe (First Component)

The solution containing the luminescent probe 1 was put into all the columns 1 of the microwell structure parts (22) disposed in 9 rows and 16 columns of the microarray device (100). The solution containing the luminescent probe 2 was put into the row 2, and similarly, the solution containing the corresponding luminescent probe was injected up to the row 16. For the ejection of the luminescent probe, a piezoelectric inkjet printer head “KM1024a” manufactured by Konica Minolta, Inc. was used.

### (3) Acquisition of Luminescent Image (First Signal Information)

The power receiving antenna of each of the microarray devices (100) was held over a USB-powered non-contact IC-card reader PaSoRi RC S300 (manufactured by Sony Corporation) to cause the microdot light emitting part to emit light. An image was captured from the top surface of the microarray section of the microarray device (100) using a microscope, and an image A in the PNG format was acquired.

The outline of the outer periphery of each microwell structure part was detected from the image A using OpenCV of an open source image processing library, and RGB data (first signal information) of the center of gravity of the detected outline was acquired.

### (4) Dripping of Specimen (Target Substance (Second Component))

Wine 1 (second component) was dropped as a specimen (target substance) so that the entire microwell structure part (22) of the microarray device (100) on which the above-described first component was disposed was covered.

### (5) Acquisition of Luminescent Image (Second Signal Information)

The power receiving antenna of each of the microarray devices (101) was held over a USB-powered non-contact IC-card reader PaSoRi RC S300 (manufactured by Sony Corporation) to cause the microdot light emitting part to emit light. An image was captured from the top surface of the microarray section of the microarray device (100) using a microscope, and an image B in the PNG format was acquired.

The outline of the outer periphery of each microwell structure part was detected from the image B using OpenCV of an open source image processing library, and RGB data (second signal information) of the center of gravity of the detected outline was acquired.

### (6) Acquiring Analysis Data of the First Signal Information and the Second Signal Information

Analysis data was obtained by subtracting the RGB data of the image B (second signal information) from the RGB data of the image A (first signal information).

### (7) Acquisition of Analysis Data Related to Other Specimens (Target Substance)

In the same manner as described above, microarray devices (100) were further produced in units of 94. Then, wines (first components) of specimens (target substances) were changed to wines 2 to 95 described in Table 4 and similarly, analytical date was obtained as follows.

TABLE 4

| | Production |
|---|---|
| Specimen wine | district label |
| 1 to 12 | SB |
| 13 to 35 | DW |
| 36 to 41 | KS |
| 42 to 51 | CH |
| 52 to 71 | PN |
| 72 to 83 | ML |
| 84 to 95 | MBA |

### (8) Analysis Method

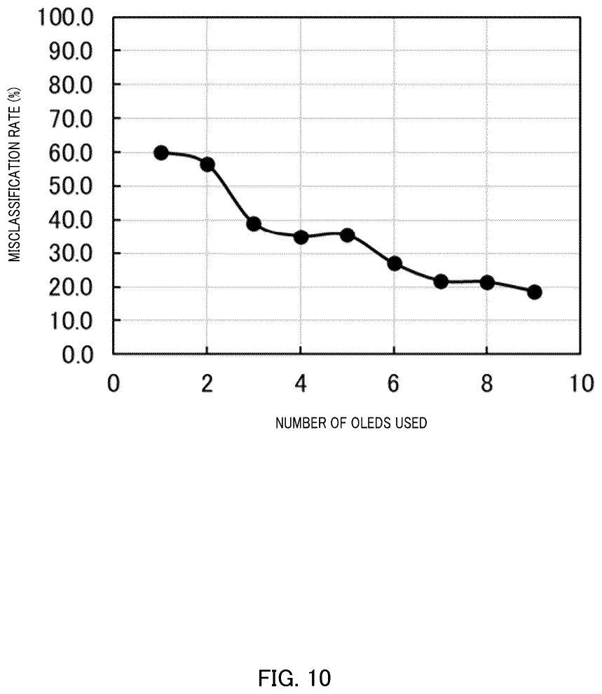

Each of the R difference value, the G difference value, and the B difference value of the analysis data was defined as a covariate. From the covariate, the misclassification rate of the place of production was obtained for each number of used microdot light emitting parts with each sample product district of wine described in Table 4 as a category. The results are illustrated in FIG. 10. From this result, it was possible to show that it is possible to reduce the misclassification rate, that is, to improve the analysis accuracy, by increasing the number of microdot light emitting parts used.

In particular, it has been found that when the number of microdot light emitting parts used is increased from one to two, and two or more of the microdot light emitting parts having emission maximum wavelengths of less than 380 nm, the microdot light emitting parts having emission maximum wavelengths of 380 nm or more and less than 500 nm, and the microdot light emitting parts having emission maximum wavelengths of 500 nm or more are prepared, the analysis accuracy is greatly improved.

Furthermore, it has been found that when the number of microdot light emitting parts used is increased from two to three and all of the microdot light emitting parts having emission maximum wavelengths of less than 380 nm, the microdot light emitting parts having emission maximum wavelengths of 380 nm or more and less than 500 nm, and the microdot light emitting parts having emission maximum wavelengths of 500 nm or more are provided, the analysis accuracy is more greatly improved.

According to the microarray device, a high-density and energy-saving detection method using a CMOS sensor or the like of a general-purpose camera can be used to analyze the state of interaction between a specimen (target substance) and a luminescent probe in a multidimensional manner.

### 4. [Example 4]

### 4-1. Synthesis of Luminescent Probes 1 to 60

### (1) Synthesis of Monomer 3

Based on the following reaction formula, a monomer 3 having a main chain containing a phosphate ester and a luminophore bonded to the main chain was synthesized via intermediates 7 to 11.

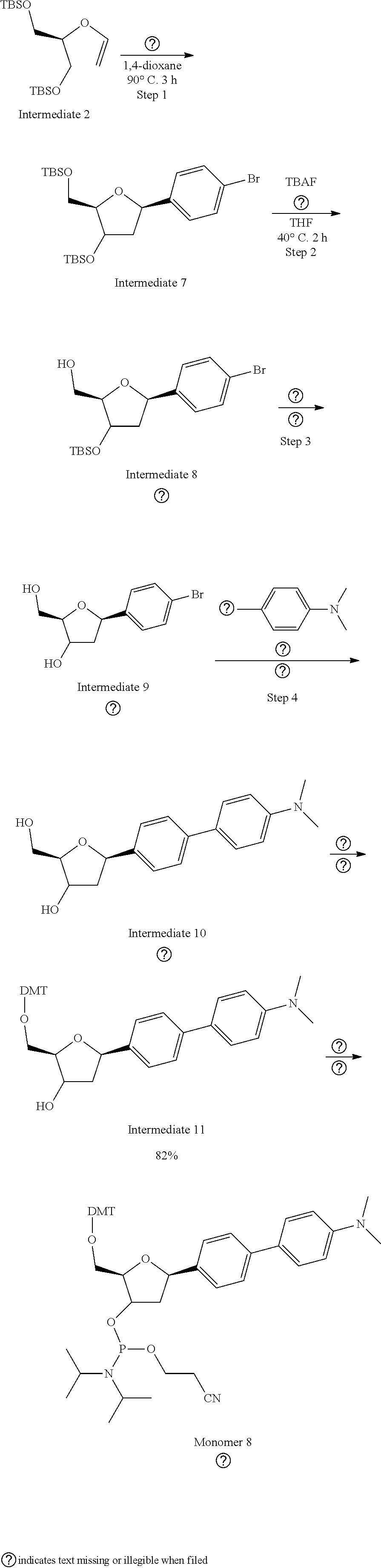

### Synthesis of Intermediate 8

A mixture of the above described intermediate 2 (2.00 g, 5.80 mmol), 1,4-dibromobenzene (8.24 g, 29.0 mmol), tris (dibenzylideneacetone) dipalladium (0) (133 mg, 145 mol), tri-tert-butylphosphonium tetrafluoroborate (168 mg, 580 mol), dicyclohexylmethylamine (1.85 mL, 8.70 mmol), 1,4-dioxane (fso) was heated at 90° C. for 3 hours. 29 mL Water was added to stop the reaction, and liquid separation and extraction were performed with ethyl acetate. The obtained organic phase was dried over magnesium sulfate, and the solvent was distilled off to obtain a crude product containing intermediate 7, which was used as it was for the next reaction.

To the crude product containing intermediate 7 were added THF 29 mL, 1 M tetrabutylammonium fluoride THF solutions (23.2 mL, 23.2 mmol), and acetic acids (1.32 mL, 23.2 mmol), and the mixture was stirred at 40° C. for 2 hr. Water was added to stop the reaction, and liquid separation and extraction were performed with ethyl acetate. The crude product obtained by distilling off the solvents was purified by silica gel column chromatography to obtain the desired intermediate 8 as a yellowish brown oil (1.08 g, 69%).

### Synthesis of Intermediate 9

Solutions of sodium triacetylborate (2.52 g, 11.9 mmol) and acetic acids (1.82 mL, 31.8 mmol) in acetonitrile 20 mL were cooled to 0° C., and solutions of the intermediates 8 (1.08 g, 3.98 mmol) in THF (13 mL) were added dropwise. After completion of the dropwise addition, the mixture was warmed to room temperature and stirred for 2 hours and 30 minutes, and then water was added to stop the reaction. Liquid separation and extraction were performed with ethyl acetate, the obtained organic phase was dried over magnesium sulfate, and the solvent was distilled off to obtain a crude product. After washing with heptane, purification by recrystallization from ethyl acetate gave the desired intermediate 9 as a colorless solid (481 mg, 44%).

### Synthesis of Intermediate 10

A mixture of intermediate 9 (1.00 g, 3.66 mmol), N,N-dimethyl-4v (4, 4, 5,5-tetramethyl-1, 3,2-dioxaborolan-2-yl) aniline (905 mg, 3.66 mmol), bis(dibenzylideneacetone) palladium (0) (106 mg, 184 μmol), ethyldiisopropylamine 2-dicyclohexylphosphino-2′, 4′,6′-triisopropylbiphenyl (175 mg, 367 μmol), potassium phosphate (2.33 g, 367 μmol), N,N-dimethylformamide (33 ml), and waster (4 ml) was stirred at 90° C. for 1 hour, then added with water to stop the reaction, and subjected to separation and extraction with dichloromethane. The crude product obtained by distilling off the solvents was purified by silica gel column chromatography to obtain the desired intermediate 10 as a colorless solid (1.10 g, 96%).

### Synthesis of Intermediate 11

A mixture of intermediate 10 (1.10 g, 3.52 mmol), 4,4′-dimethoxytritylchloride (1.32 g, 3.90 mmol), ethyldiisopropylamine (0.92 mL, 5.29 mmol), and dehydrated pyridine (17.5 mL) was stirred at room temperature for 4 hours, and then methanol was added thereto to stop the reaction. The solvents were distilled off and the obtained crude product was purified by silica gel column chromatography to obtain the desired intermediate 11 as a yellow viscous solid (2.89 g, 82%).

### Synthesis of Monomer 3

To a mixture of intermediate 11 (1.23 g, 2.00 mmol), ethyldiisopropylamine (1.39 mL, 8.00 mmol) and dehydrated dichloromethane (80 mL) was added dropwise 2-cyanoethyldiisopropylchlorophosphoroamidite (468 μL, 2.10 mmol) at room temperature. After stirring for 2 hours, the solvent was distilled off to obtain a crude product. Purification by silica gel column chromatography gave the target monomer 3 as a yellow viscous solid (1.53 g, 94%).

### (2) Preparation of Monomer 4

Monomer 4, a reagent of the structure shown below, was purchased from Glen Research (Sterling, Va). Thymidine contained in the monomer 4 is a type of natural base.

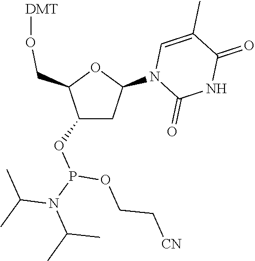

### (3) Synthesis of Monomer 5

Monomer 5 was synthesized according to a non-patent document (J. Am. Chem. Soc. 1996, 118, 7671-7678). Note that monomer 5 is a structural isomer of the above-described monomer 1 and is a monomer in which the sugar structure is an α-form.

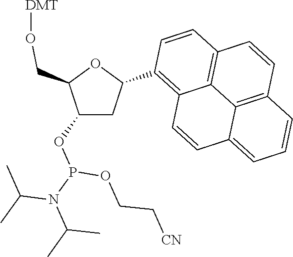

### (4) Synthesis of Luminescent Probes 1 to 60

### (4-1) Preparation of Light Emitting Probes 1 to 16

Light emitting probes 1 to 16 were prepared as in Example 1 described above.

### (4-2) Synthesis of Luminescent Probe 1, and 17 to 31

As illustrated in Table 5 below, 16 types of mixed-sequence oligonucleotides (Seq. 1 and 17 to 31) with the monomer 1 and the aniline-containing monomer 3 described above were synthesized by a method similar to the method for synthesizing the luminescent probes 1 to 16 described above. It was confirmed that the luminescent probes 1 and 17 to 31 emitted fluorescence and exciplex light emission, respectively, by specific excitation light (light having wavelengths 350 nm).

TABLE 5

| | | Luminescent dye molecules | | | | | | | | | | | | | | | |
|---|---|---|---|---|---|---|---|---|---|---|---|---|---|---|---|---|---|
| | | 1 | 17 | 18 | 19 | 20 | 21 | 22 | 23 | 24 | 25 | 26 | 27 | 28 | 29 | 30 | 31 |
| Monomer | 5′ | 1 | 1 | 1 | 1 | 1 | 1 | 1 | 1 | 3 | 3 | 3 | 3 | 3 | 3 | 3 | 3 |
| sequence | 3′ | 1 | 1 | 1 | 1 | 3 | 3 | 3 | 3 | 1 | 1 | 1 | 1 | 3 | 3 | 3 | 3 |
| | | 1 | 1 | 3 | 3 | 1 | 1 | 3 | 3 | 1 | 1 | 3 | 3 | 1 | 1 | 3 | 3 |
| | | 1 | 3 | 1 | 3 | 1 | 3 | 1 | 3 | 1 | 3 | 1 | 3 | 1 | 3 | 1 | 3 |

| | | | | | | | | | | | | | | | | |
|---|---|---|---|---|---|---|---|---|---|---|---|---|---|---|---|---|
| Proportion of β- | 100 | 100 | 100 | 100 | 100 | 100 | 100 | 100 | 100 | 100 | 100 | 100 | 100 | 100 | 100 | 100 |
| form of deoxyribose | | | | | | | | | | | | | | | | |
| (%) | | | | | | | | | | | | | | | | |
| Number of | 4 | 4 | 4 | 4 | 4 | 4 | 4 | 4 | 4 | 4 | 4 | 4 | 4 | 4 | 4 | 4 |
| chromophores or | | | | | | | | | | | | | | | | |
| luminophores | | | | | | | | | | | | | | | | |
| Proportion of | 0 | 0 | 0 | 0 | 0 | 0 | 0 | 0 | 0 | 0 | 0 | 0 | 0 | 0 | 0 | 0 |
| natural bases (%) | | | | | | | | | | | | | | | | |

### (4-3) Synthesis of Luminescent Probe 1, and 32 to 45

As illustrated in Table 6 below, 15 types of oligonucleotides (Seq1 and Seq32 to 45) having a mixed sequence of the monomer 1 and the thymidine-containing monomer 4 described above were synthesized by a method similar to the method for synthesizing the luminescent probes 1 to 16 described above. In addition, it was confirmed that the luminescent probes 1 and 32 to 45 emitted fluorescence and excimer light, respectively, by specific excitation light (light of wavelengths 350 nm).

TABLE 6

| | | Luminescent dye molecules | | | | | | | | | | | | | | |
|---|---|---|---|---|---|---|---|---|---|---|---|---|---|---|---|---|
| | | 1 | 32 | 33 | 34 | 35 | 36 | 37 | 38 | 39 | 40 | 41 | 42 | 43 | 44 | 45 |
| Monomer | 5′ | 1 | 1 | 1 | 1 | 1 | 1 | 1 | 1 | 4 | 4 | 4 | 4 | 4 | 4 | 4 |
| sequence | 3′ | 1 | 1 | 1 | 1 | 4 | 4 | 4 | 4 | 1 | 1 | 1 | 1 | 4 | 4 | 4 |
| | | 1 | 1 | 4 | 4 | 1 | 1 | 4 | 4 | 1 | 1 | 4 | 4 | 1 | 1 | 4 |
| | | 1 | 4 | 1 | 4 | 1 | 4 | 1 | 4 | 1 | 4 | 1 | 4 | 1 | 4 | 1 |

| | | | | | | | | | | | | | | | |
|---|---|---|---|---|---|---|---|---|---|---|---|---|---|---|---|
| Proportion of β- | 100 | 100 | 100 | 100 | 100 | 100 | 100 | 100 | 100 | 100 | 100 | 100 | 100 | 100 | 100 |
| form of | | | | | | | | | | | | | | | |
| deoxyribose (%) | | | | | | | | | | | | | | | |
| Number of | 4 | 3 | 3 | 2 | 3 | 2 | 2 | 1 | 3 | 2 | 2 | 1 | 2 | 1 | 1 |
| chromophores or | | | | | | | | | | | | | | | |
| luminophores | | | | | | | | | | | | | | | |
| Proportion of | 0 | 25 | 25 | 50 | 25 | 50 | 50 | 75 | 25 | 50 | 50 | 75 | 50 | 75 | 75 |
| natural bases (%) | | | | | | | | | | | | | | | |

### (4-4) Synthesis of Light Emitting Probes 46 to 60

As illustrated in Table 7 below, 15 types of oligonucleotides (Seq46 to 60) having a mixed sequence of the monomer 5 and the monomer 2 each containing an α-form sugar structure were synthesized by a method similar to the method for synthesizing the above described luminescent probes 1 to 16. It was confirmed that the light emitting probes 46 to 60 emit fluorescence and excimer light, respectively, by specific excitation light (light having a wavelength of 350 nm).

TABLE 7

| | | Luminescent dye molecules | | | | | | | | | | | | | | |
|---|---|---|---|---|---|---|---|---|---|---|---|---|---|---|---|---|
| | | 46 | 47 | 48 | 49 | 50 | 51 | 52 | 53 | 54 | 55 | 56 | 57 | 58 | 59 | 60 |
| Monomer | 5′ | 5 | 5 | 5 | 5 | 5 | 5 | 5 | 5 | 2 | 2 | 2 | 2 | 2 | 2 | 2 |
| sequence | 3′ | 5 | 5 | 5 | 5 | 2 | 2 | 2 | 2 | 5 | 5 | 5 | 5 | 2 | 2 | 2 |
| | | 5 | 5 | 2 | 2 | 5 | 5 | 2 | 2 | 5 | 5 | 2 | 2 | 5 | 5 | 2 |
| | | 5 | 2 | 5 | 2 | 5 | 2 | 5 | 2 | 5 | 2 | 5 | 2 | 5 | 2 | 5 |

| | | | | | | | | | | | | | | | |
|---|---|---|---|---|---|---|---|---|---|---|---|---|---|---|---|
| Proportion of β-form | 0 | 0 | 0 | 0 | 0 | 0 | 0 | 0 | 0 | 0 | 0 | 0 | 0 | 0 | 0 |
| of deoxyribose (%) | | | | | | | | | | | | | | | |
| Number of | 4 | 3 | 3 | 2 | 3 | 2 | 2 | 1 | 3 | 2 | 2 | 1 | 2 | 1 | 1 |
| chromophores or | | | | | | | | | | | | | | | |
| luminophores | | | | | | | | | | | | | | | |
| Proportion of natural | 0 | 0 | 0 | 0 | 0 | 0 | 0 | 0 | 0 | 0 | 0 | 0 | 0 | 0 | 0 |
| bases (%) | | | | | | | | | | | | | | | |

### 4-2. Analysis Using Luminescent Probe

### (1) Analysis Using Luminescent Probes 1 to 15

### Light-Emitting Probe Disposing Step

A plurality of 96-well microplates in which wells having 7 mm opening diameters were disposed at 9 mm intervals in 12 columns and ×8 rows were prepared. In the 96-well microplate, the luminescent probes 1 to 15 described above were disposed in an amount of 100 μl per well, the number of the probes being the same as the number of the target substances, with an automatic dispenser (NichiMart CUBE, manufactured by NICHIRYO Co., Ltd) to form a plurality of reaction fields.

### First Signal Information Acquiring Step

A fluorescence spectrum obtained when a 96-well microplate in which the above-described luminescent probes were disposed was irradiated with excitation light (wavelengths 350 nm) was acquired as first signal information.

### Target Substance Disposing Step

To the 96-well microplate after the first signal information acquiring step, 7 types and 95 brands of beverages (type I:) 12 BRAND, TYPE II: 23 BRAND, TYPE III: 6 BRAND, TYPE IV: 10 BRAND, TYPE V: 20 BRAND, TYPE VI: 13 BRAND, TYPE VII: Eleven brands) were disposed in 20 μl portions by the same method as described above.

### Second Signal Information Acquiring Step

The fluorescence spectra when the 96-well microplate in which the first components and the second components were disposed was irradiated with the excitation light (wave length 350 nm) were acquired as the second signal information.

### Analysis Step

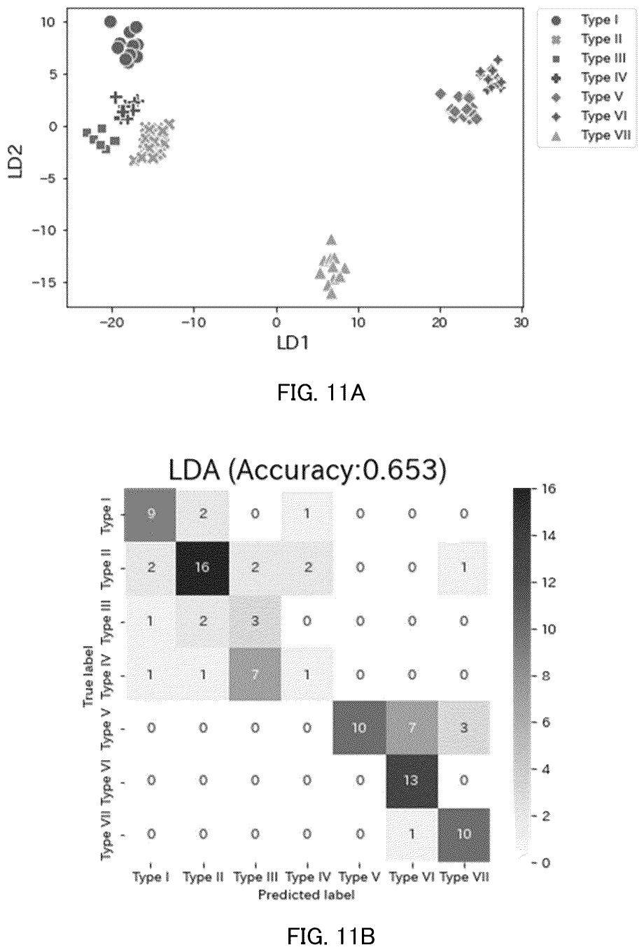

The analysis data was calculated by subtracting the first signal information acquired in the first signal information acquiring step from the second signal information acquired in the second signal information acquiring step. Then, learning was performed using the analysis data as explanatory variables and the classification data of each beverage as objective variables, and a discriminant model was created by linear discriminant analysis (LDA). The obtained linear discriminant analysis model plots are illustrated in the 11A of the figure. Thereafter, calculation of a correct answer rate and creation of a confusion matrix were performed by cross-validation of 6 divisions, and generalization performance of the discriminant model was quantified. The confusion matrix is illustrated in FIG. 11B.

### Results

As illustrated in FIG. 11B, the discriminant model was able to classify the types of 95 brands of beverages with an accuracy of about 65%.

### (2) Analysis Using Luminescent Probes 1 and 17 to 31

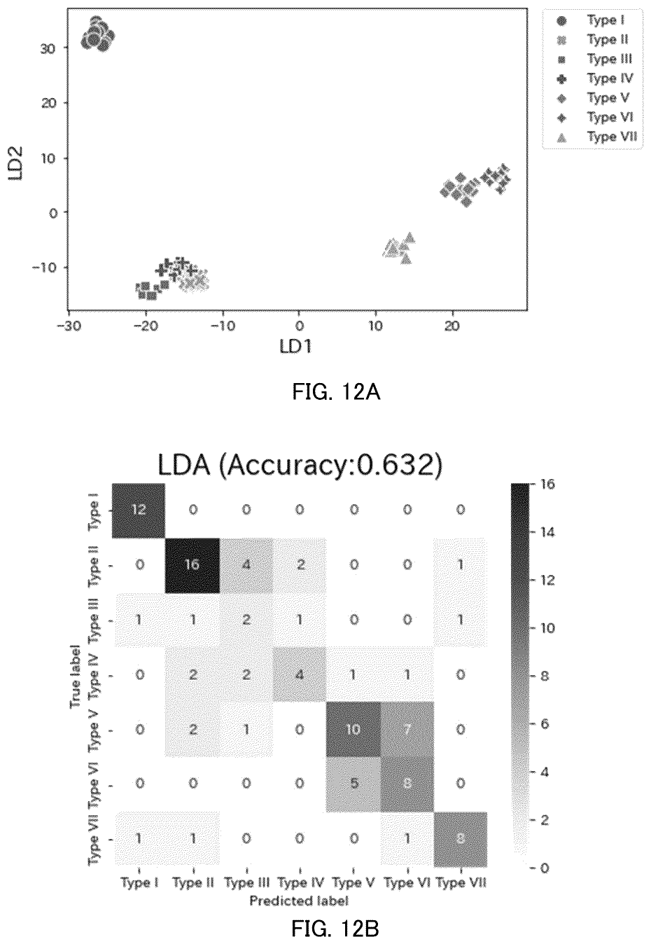

Using the luminescent probes 1 and 17 to 31 (16 types), the steps from the luminescent probe disposing step to the analysis step described above were performed in the same manner. The obtained linear discriminant analysis model plots are illustrated in the 12A of the figure. Then, calculation of a correct answer rate and creation of a confusion matrix were performed by cross-validation of six divisions, and generalization performance of the discriminant model was quantified. The confusion matrix is illustrated in FIG. 12B.

### Results

As illustrated in FIG. 12B, the discriminant model was able to classify the types of 95 brands of beverages with an accuracy of about 63%.

### (3) Analysis Using Luminescent Probes 1 to 15 and 17 to 31

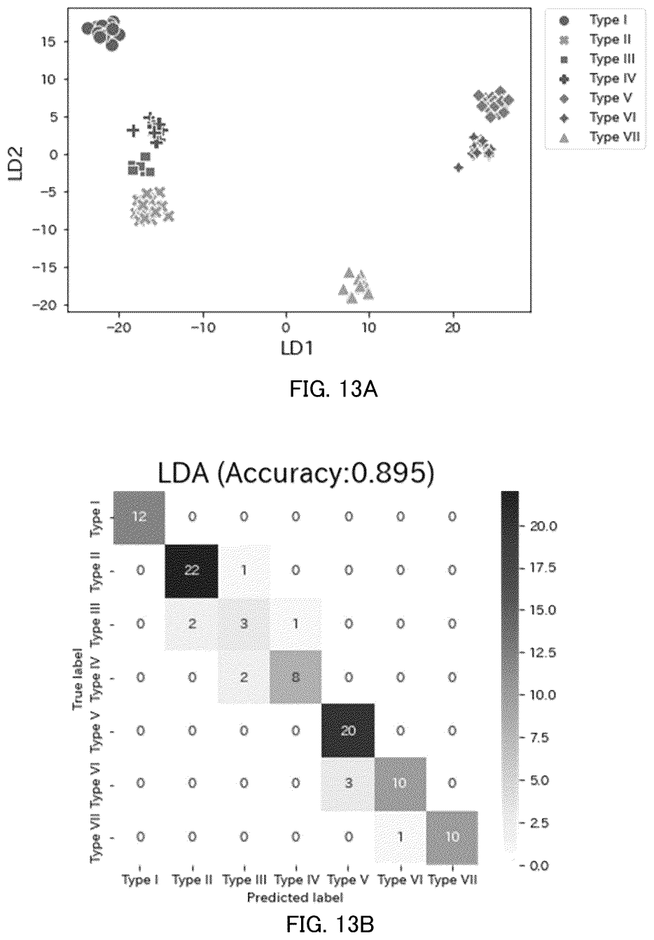

Using the luminescent probes 1 to 15 and 17 to 31 (30 types), the steps from the luminescent probe disposing step to the analysis step described above were performed in the same manner. The linear discriminant analysis model plots thus obtained are illustrated in the 13A of the drawing. Then, calculation of a correct answer rate and creation of a confusion matrix were performed by cross-validation of six divisions, and generalization performance of the discriminant model was quantified. The confusion matrix is illustrated in FIG. 13B.

### Results

As illustrated in 13B of the figure, the discriminant model was able to classify the types of the 95 brands of beverages with an accuracy of about 90%.

### (4) Analysis Using Luminescent Probes 1 and 32 to 45

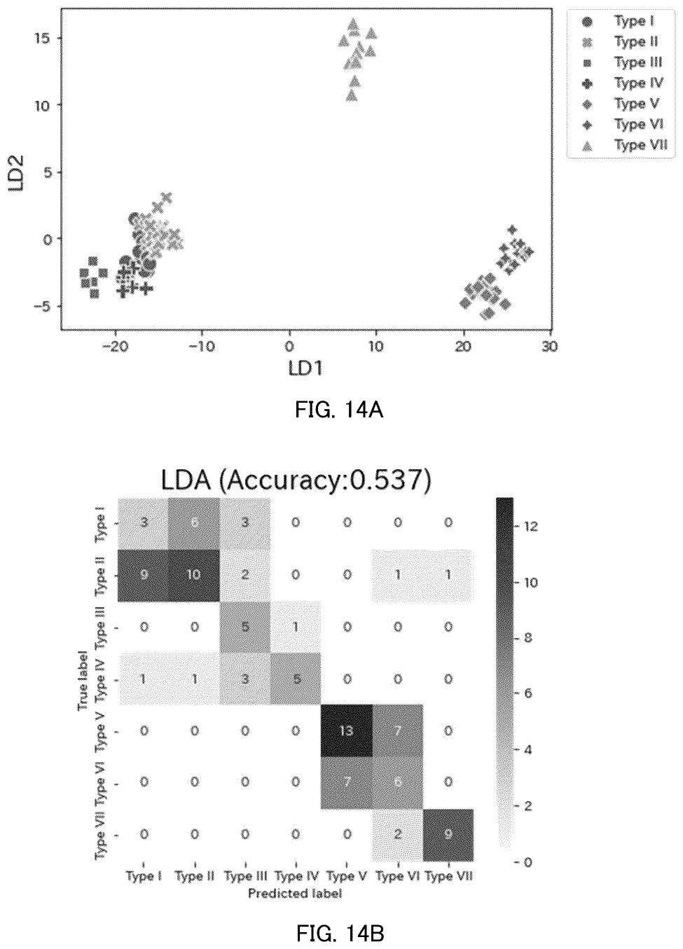

Using the luminescent probes 1 and 32 to 45 (15 types), the steps from the luminescent probe disposing step to the analysis step described above were performed in the same manner. The linear discriminant analysis model plots thus obtained are illustrated in the 14A of the drawing. Then, calculation of a correct answer rate and creation of a confusion matrix were performed by cross-validation of six divisions, and generalization performance of the discriminant model was quantified. The confusion matrix is illustrated in FIG. 14B.

### Results

As illustrated in FIG. 14B, the discriminant model was able to classify the types of 95 brands of beverages with an accuracy of about 54%.

### (5) Analysis Using Luminescent Probes 46 to 60

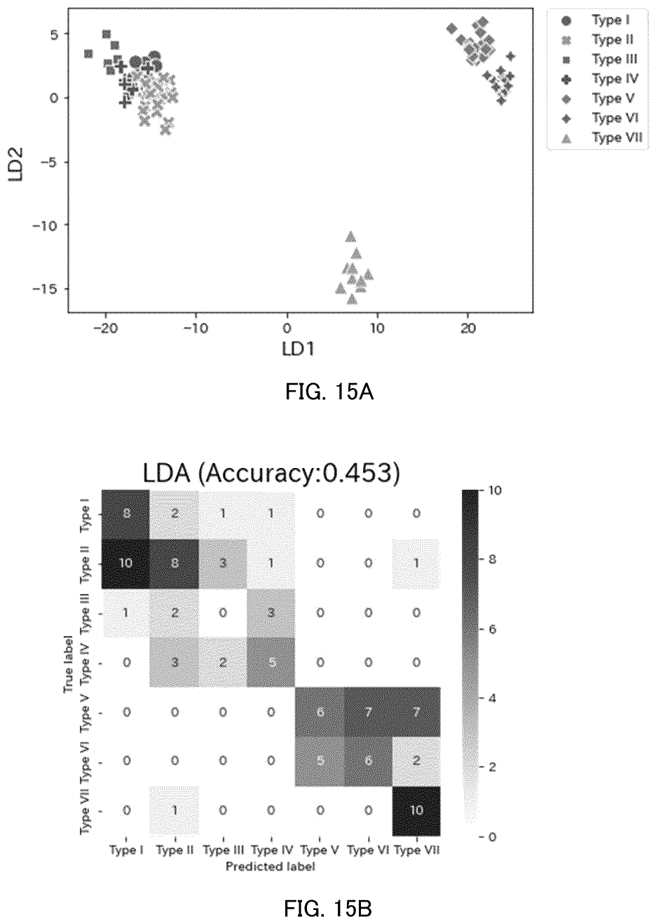

Using the light emitting probes 46 to 60 (15 types), the steps from the light emitting probe disposing step to the analysis step described above were performed in the same manner. The linear discriminant analysis model plots thus obtained are illustrated in the 15A of the drawing. Then, calculation of a correct answer rate and creation of a confusion matrix were performed by cross-validation of six divisions, and generalization performance of the discriminant model was quantified. The confusion matrix is illustrated in FIG. 15B.

### Results

As illustrated in FIG. 15B, the discriminant model was able to classify the types of 95 brands of beverages with an accuracy of about 45%. The reason why the accuracy was lower than those of the other analyses can be exemplified by a small proportion of the β-form sugar structure in the luminescent probe.

(6) Analysis when Explanatory Variables are Randomly Replaced

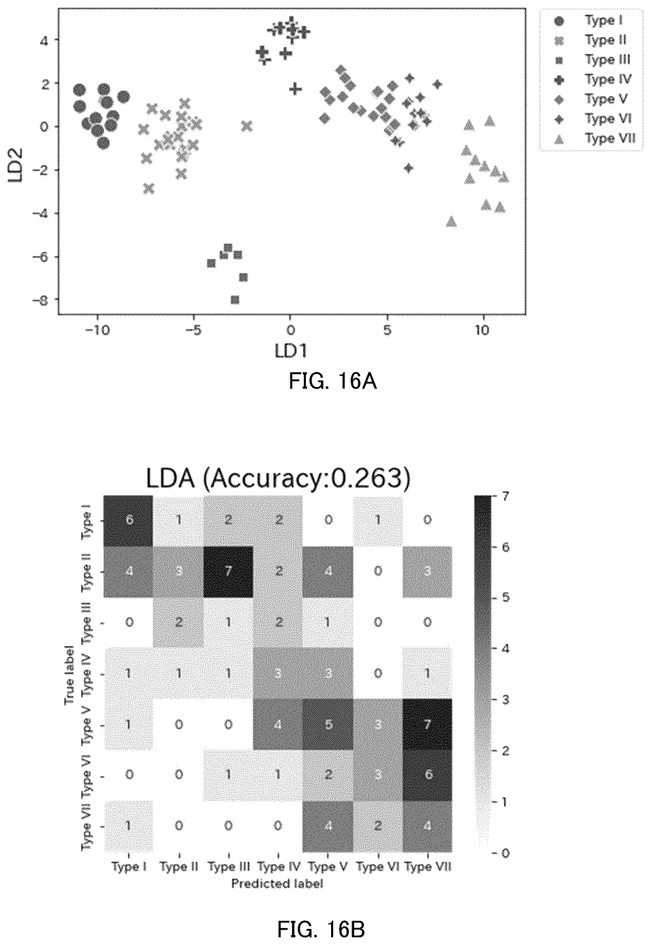

In order to clarify the effect of the present invention, a model was created by randomly replacing explanatory variables as a negative control experiment. Specifically, the explanatory variables obtained by performing the above-described measurement and analysis steps with the luminescent probes 1 to 15 (15 types) were randomly replaced to create a discriminant model. The obtained linear discriminant analysis model plots are illustrated in the 16A of the figure. Then, calculation of a correct answer rate and creation of a confusion matrix were performed by cross-validation of six divisions, and generalization performance of the discriminant model was quantified. The confusion matrix is illustrated in FIG. 16B.

### Results

As illustrated in FIG. 16B, the discriminant model in which the explanatory variables were randomly replaced was able to classify the types of 95 brands of beverages at about 25%. The results indicate that the above-described accuracy is not obtained by chance in a discriminant model in which the above-described luminescent probe is used and explanatory variables and an objective variable are correctly set. From the above results, it is understood that various compounds can be analyzed with high accuracy according to the discriminant model in which the explanatory variable and the objective variable are correctly set using the luminescent probe.

### 5 [Example 5]

### [Preparation of Luminescent Probe Containing Liquids 1 to 16]

The luminescent probes 1 to 16 were respectively dissolved in solutions containing Na₂HPO₄/NaH₂PO₄ (150 mM) and NaCl (50 mM), and having a pH level of 8.5 and a Tween20 concentration of 0.01% to prepare luminescent probe-containing solutions 1 to 16.

### [Production of Microarray]

The luminescent probe-containing solutions 1 to 16 were spotted on 3DNHS slides made of PolyAn at 20 to 24° C. and 70% RH. A humidity chamber was used for immobilization and rehydration of the spotted luminescent probe-containing solutions 1 to 16. The humidity chamber was filled with 1×SSC at 50 to 100 ml, the spotted glass slide was placed in the chamber, and the spots were allowed to rehydrate for 24 hours.

Next, the slide glass was blocked with ethanol amine (50 mM) and Tris (100 mM) in pH9 for 2 hours. The glass slides were washed with solutions containing NaCl (137 mM), KCl (2.7 mM), Na₂HPO₄ (4.3 mM), and KH₂ PO₄ (14 mM), having a pH 7.5 and a concentration of Tween 20 of 0.05%. Furthermore, the slide glass was washed with solutions containing NaCl (137 mM), KCl (2.7 mM), Na₂HPO₄ (4.3 mM), and KH₂PO₄ (14 mM), and having a pH of 7.5.

The slide glass was dried by rotation in a centrifuge (1000 rpm) for 3 minutes to produce a microarray on which each of the luminescent probes 1 to 16 was immobilized. Using the microarray, in the same manner as in Example 3 described above, wine was dropped as a specimen (target substance), and the same analysis was performed from the RGB values of the obtained luminescent image. It was possible to determine each sample product district of wine as a category.

This application claims the benefit of Japanese Patent Application No. 2022-037178, filed Mar. 10, 2022. The contents described in the specification of the application and the drawings are all incorporated into {the specification of the present application.

### INDUSTRIAL APPLICABILITY

According to the analysis method and the analysis system described above, it is possible to easily analyze the target substance by using data obtained from a plurality of types of reactions including the interaction of the first component and/or the second component. Furthermore, it is also possible to collectively and simply acquire a large amount of multidimensional data. Therefore, the present invention is very useful for analysis in various fields such as a medical field, an industrial field, and a food field.

In addition, the luminescent probe exhibiting a plurality of types of luminescence or the carrier containing the same can be used as an indicator for inspection by being brought into a solution state or a dispersion liquid state, and furthermore, it becomes possible to acquire a large amount of data in a short time by utilizing an inkjet, an automatic dispensing machine or the like, which can greatly contribute to the activation and acceleration of industries such as data driven research and development like an inverse problem solving method and data driven inspection and diagnosis.

Furthermore, by using the fluorescent dye molecule of the present invention as an immobilized measurement chip, the portability is enhanced and the restriction on the place where it is used is almost eliminated.

The present invention can generate a large amount of real data highly compatible with machine learning and deep learning only by measuring light and color.

Furthermore, it is also characterized in that an expensive and large-sized instrumental analysis section is not required, and from the various characteristics as described above, it becomes possible to acquire data by bringing it to various working sites such as medical sites using liquid substances such as blood and saliva as a food source, food processing sites such as alcoholic beverages and fruit juice, production sites and water treatment plants in the chemical industry requiring sewage treatment, and further pastures for collecting milk and raw milk, so that it is expected to develop into a new technology which also matches the digital rural urban national concept proposed by the Japanese government.

### REFERENCE SIGNS LIST

1 Support
10 Light source unit
11 Anode
12 Insulating part
13. Microdot light emitting part
14 Cathode
15 Sealing member
16 Barrier material
17 NFC Tag
18 Power receiving section
19 Receiving layer
20 Multi-well unit
21 Cured layer
22 Microwell structure part
100 Microarray device

1. An analysis system, comprising:
a plate comprising a plurality of reaction fields for accommodating a first component and a second component, the plurality of reaction fields being partitioned at intervals;
a signal information acquirer configured to acquire first signal information from the plate in a case where the first component is accommodated in the plurality of reaction fields, and second signal information from the plate in a case where the first component and the second component are accommodated such that the second component is further accommodated in the plurality of reaction fields from which the first signal information has been acquired; and
an analyzer for performing machine learning and analyzing a difference between the first signal information and the second signal information,
wherein
at least two of the plurality of reaction fields are regions for respectively accommodating a plurality of the first components comprising different compositions, and
at least one of the plurality of reaction fields is a region for causing a plurality of types of reactions comprising an interaction of the first component and/or the second component.

2. The analysis system according to claim 1, wherein
the plurality of types of reactions are interactions that non-specifically occur between the first component and the second component.

3. The analysis system according to claim 1, wherein
the plurality of types of reactions are a plurality of types of light-emitting reactions caused by an interaction of the first component and/or the second component.

4. The analysis system according to claim 1, wherein
in the plate, the first component is immobilized in the plurality of reaction fields.

5. The analysis system according to claim 1, further comprising:
an inkjet printer for applying the first component and the second component to the plurality of reaction fields.

6. The analysis system according to claim 1, wherein
the first signal information and the second signal information are fluorescent fingerprints.

7. The analysis system according to claim 1, wherein
the first component comprises a luminescent probe, and the second component comprises a target substance that is an analysis target.

8. The analysis system according to claim 7, wherein:
the luminescent probe comprises
a nucleic acid structure, and
at least one of chromophore or luminophore bonded to a main chain of the nucleic acid structure,
wherein
for a single excitation light, two or more types of light selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregated organic luminescence are emitted.

9. The analysis system according to claim 8, wherein
the nucleic acid structure is a structure derived from at least one compound selected from the group consisting of DNA, RNA, phosphorothioate oligodeoxynucleotide, 2′-O-(2-methoxy) ethyl-modified nucleic acid, siRNA, crosslinked nucleic acid, peptide nucleic acid, aTNA, SNA, GNA, LNA, and morpholino antisense nucleic acid.

10. The analysis system according to claim 8, wherein
the main chain of the nucleic acid structure comprises
at least one structural unit comprising a sugar structure derived from pentose or hexose, and a phosphate ester bond bonded to the sugar structure.

11. The analysis system according to claim 1, wherein
the analyzer extracts RGB data or hyperspectral data from digital image data acquired as the first signal information and the second signal information.

12. A plate to be used for machine learning, comprising:
a plurality of reaction fields for accommodating a first component and a second component, wherein
the plurality of reaction fields are partitioned at intervals,
at least two of the plurality of reaction fields are regions for respectively accommodating a plurality of the first components comprising different compositions, and
at least one of the plurality of reaction fields is a region for causing a plurality of types of reactions comprising an interaction of the first component and/or the second component.

13. The plate according to claim 12, wherein
the first component comprises
a nucleic acid structure, and
at least one of chromophore or luminophore bonded to a main chain of the nucleic acid structure,
wherein
for a single excitation light, two or more types of light selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregated organic luminescence are emitted.

14. The plate according to claim 13, wherein
the nucleic acid structure is a structure derived from at least one compound selected from the group consisting of DNA, RNA, phosphorothioate oligodeoxynucleotide, 2′-O-(2-methoxy) ethyl-modified nucleic acid, siRNA, crosslinked nucleic acid, peptide nucleic acid, aTNA, SNA, GNA, LNA, and morpholino antisense nucleic acid.

15. The plate according to claim 13, wherein
the main chain of the nucleic acid structure comprises
at least one structural unit comprising a sugar structure derived from pentose or hexose, and a phosphate ester bond bonded to the sugar structure.

16. An analysis method, comprising:
disposing a first component in each of a plurality of reaction fields of a plate, the plate comprising the plurality of reaction fields for accommodating the first component and a second component, the plurality of reaction fields being partitioned from each other at intervals;
acquiring first signal information from the plate in which the first component is disposed;
further disposing the second component in each of the plurality of reaction fields of the plate from which the first signal information has been acquired;
acquiring second signal information from the plate in which the first component and the second component are disposed; and
machine learning and analyzing a difference between the first signal information and the second signal information,
wherein
in the disposing the first component, a plurality of the first components comprising different compositions are disposed in at least two of the plurality of reaction fields, and
in the disposing the second component, a plurality of types of reactions comprising an interaction of the first component and/or the second component are caused in at least one of the plurality of reaction fields.

17. The analysis method according to claim 16, wherein
the plurality of types of reactions are interactions that non-specifically occur between the first component and the second component.

18. The analysis method according to claim 17, wherein
the plurality of types of reactions are a plurality of types of light-emitting reactions caused by an interaction of the first component and/or the second component.

19. The analysis method according to claim 16, wherein
the first signal information and the second signal information are fluorescent fingerprints.

20. The analysis method according to claim 16, wherein
in the disposing the second component, the second component is disposed in the reaction field by an inkjet method.

21. The analysis method according to claim 16, wherein
the first component comprises a luminescent probe and the second component comprises a target substance.

22. The analysis method according to claim 21, wherein
the luminescent probe comprises
a nucleic acid structure, and
at least one of chromophore or luminophore bonded to a main chain of the nucleic acid structure,
wherein
for a single excitation light, two or more types of light selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregated organic luminescence are emitted.

23. The analysis method according to claim 22, wherein
the nucleic acid structure is a structure derived from at least one compound selected from the group consisting of DNA, RNA, phosphorothioate oligodeoxynucleotide, 2′-O-(2-methoxy) ethyl-modified nucleic acid, siRNA, crosslinked nucleic acid, peptide nucleic acid, and morpholino antisense nucleic acid.

24. The analysis method according to claim 22, wherein
the nucleic acid structure comprises
a main chain comprising at least one structural unit comprising a sugar structure derived from pentose or hexose, and a phosphate ester bond bonded to the sugar structure.
